(19) United States
(12) Patent Application Publication
(10) Pub. No.: US 2003/0019049 A9
(43) Pub. Date: Jan. 30, 2003

(54) Compositions for oxidation dyeing keratinous fibers comprising at least one 1-(4-aminophenyl) pyrrolidine and at least one cationic polymer,and dyeing methods

(76) Inventors: Sylvain Kravtchenko, Asnieres (FR); Alain Lagrange, Coupvray (FR)

Correspondence Address: FINNEGAN, HENDERSON, FARABOW, GARRETT &, DUNNER LLP, 1300 I STREET, NW, WASHINGTON, DC, 20006, US

(21) Appl. No.: 09/836,703

(22) Filed: Apr. 18, 2001

(30) Foreign Application Priority Data
Apr. 18, 2000 (FR) 00 04993

(51) Int. Cl. A61K 7/13

(52) U.S. Cl. 8/405; 8/406; 8/421

(57) ABSTRACT

Compositions for oxidation dyeing keratinous fibers, for example, human keratinous fibers such as hair, comprising, in a medium suitable for dyeing, at least one oxidation dye precursor chosen from 1-(4-aminophenyl)-pyrrolidines and acid addition salts thereof, and at least one cationic polymer. Dyeing methods and devices using said compositions.

The present invention relates to compositions for oxidation dyeing keratinous fibers, for example, human keratinous fibers such as hair, comprising, in a medium suitable for dyeing, at least one oxidation dye precursor chosen from 1-(4-aminophenyl)-pyrrolidines and acid addition salts thereof, and at least one cationic polymer.

It is known to dye keratinous fibers, for example, human hair, with dyeing compositions comprising oxidation dye precursors, generally called “oxidation bases.” Representative oxidation bases include ortho- and para-phenylenediamines, ortho- and para-aminophenols, and heterocyclic bases.

Oxidation dye precursors are compounds initially only slightly colored or not colored that develop their dyeing power in the hair in the presence of oxidizing agents, leading to the formation of colored compounds. The formation of these colored compounds results either from oxidative condensation of the “oxidation bases” with themselves, or oxidative condensation of the “oxidation bases” with color-modifying compounds, or “couplers,” which are generally present in the dyeing compositions used in oxidation dyeing. Representative couplers include meta-phenylenediamines, meta-aminophenols, meta-diphenols, and certain heterocyclic compounds.

The variety of compositions that can be employed in oxidation coloration, chosen from oxidation bases, oxidation couplers and mixtures of oxidation bases and couplers, can contribute to a palette very rich in color.

It is desirable for such oxidation dyes, otherwise called “permanent” dyes, to satisfy at least one of the following—make it possible to obtain shades of the desired intensity and tend to exhibit good resistance toward at least one external agent, such as, for example, light, adverse weather conditions, washing, permanent waving, perspiration, and rubbing.

Further, it is desirable that such dyes be able to cover grey hair, and thus should be the least selective possible, that is to say they should make it possible to obtain the smallest possible differences in color all along the same keratinous fiber, which may indeed be differently sensitized (i.e. damaged) between its tip and its root.

The permanent dyeing of hair with para-phenylenediamine (PPD) coupling products in the presence of hydrogen peroxide has been known for a long time and is in very widespread use. Nevertheless, better-tolerated oxidation bases have been sought and proposed as alternatives to PPD. For example, the tertiary base N,N-bis(β-hydroxyethyl)-para-phenylenediamine has been widely used in commercial hair-dyeing products, however, the colors obtained using these compositions tend to have at least one of the following drawbacks: less intense colors, less chromatic shades, or less resistant to various external agents to which the hair may be subjected.

The inventors have discovered that it is possible to overcome at least one of the above-mentioned drawbacks. By combining at least one (as used herein, “at least one” means one or more and thus includes mixtures and combinations) oxidation base chosen from 1-(4-aminophenyl)pyrrolidines and acid addition salts thereof with at least one cationic polymer, however, the inventors have just now discovered that it is possible to obtain oxidation dyes capable of producing shades of colors that may have at least one of the following properties: shades that are varied, chromatic, intense, aesthetic, not very selective, and/or that exhibit good resistance to the various attacks to which the fibers may be subjected.

As used herein, the term “lower alkyl” means an alkyl chosen from saturated and unsaturated, branched and unbranched C₁-C₆ alkyl groups.

One subject of the invention is a composition for oxidation dyeing keratinous fibers, for example, human keratinous fibers such as hair, comprising, in a medium suitable for dyeing:

(i) at least one oxidation dye precursor chosen from 1-(4-aminophenyl)-pyrrolidines of formula (I) and acid addition salts thereof:

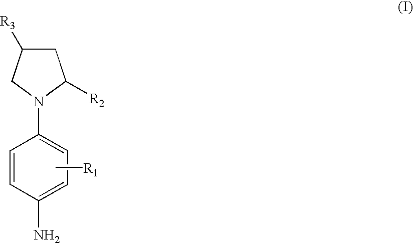

wherein:

R₁ is chosen from a hydrogen atom, C₁-C₆ alkyl groups, C₁-C₅ monohydroxyalkyl groups, and C₂-C₅ polyhydroxyalkyl groups,

R₂ is chosen from a hydrogen atom, a —CONH₂ group, C₁-C₅ monohydroxyalkyl groups, and C₂-C₅ polyhydroxyalkyl groups, and

R₃ is chosen from a hydrogen atom, and a hydroxyl group, and

(ii) at least one cationic polymer chosen from:

(1) homopolymers and copolymers comprising, as a constituent of the chain, at least one unit chosen from units formula (II):

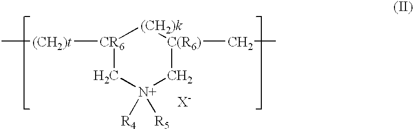

wherein:

k and t, which are identical or different, are each chosen from 0 and 1, provided that the sum of k+t is equal to 1,

R₄ and R₅, which are identical or different, are each chosen from (C₁-C₂₂) alkyl groups, (C₁-C₅)-hydroxyalkyl groups, and (C₁-C₄)amidoalkyl groups,

R₄ and R₅, together with the nitrogen cation to which they are commonly bonded, optionally form a cationic heterocyclic group chosen from a piperidinyl group and a morpholinyl group,

R₆, which are identical or different, are each chosen from a hydrogen atom and a methyl group, and

X⁻ is an anion;

(2) quaternary diammonium polymers comprising repeating units of formula (III):

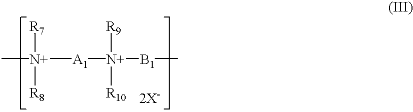

wherein:

R₇, R₈, R₉, and R₁₀, which are identical or different, are each chosen from (C₁-C₂₀) aliphatic groups, (C₃-C₂₀) alicyclic groups, (C₇-C₂₀) arylaliphatic groups, and lower hydroxyalkyl groups,

at least two of said R₇, said R₈, said R₉, and said R₁₀, together with the nitrogen cations to which they are attached, optionally form at least one cationic heterocyclic ring optionally comprising an additional heteroatom other than nitrogen,

R₇, R₈, R₉, and R₁₀, which are identical or different, are each optionally chosen from linear and branched C₁-C₆ alkyl groups substituted with at least one group chosen from nitrile groups, ester groups, acyl groups, amide groups, —CO—O—R₁₁—D groups, and —CO—NH—R₁₁—D groups, wherein R₁₁ is chosen from alkylene groups and D is chosen from quaternary ammonium groups,

A₁ and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,

X⁻ is an anion,

said A₁, said R₇, and said R₉ optionally form a piperazine ring, together with the two nitrogen cations to which they are attached, and

provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is chosen from

—(CH₂)ₙ—CO—D—OC—(CH₂)ₙ— groups, wherein:

n is an integer ranging from 1 to 100, such as, for example, from 1 to 50, and

D is chosen from:

a) glycol residues of formula: —O—Z—O—, wherein Z is chosen from linear and branched hydrocarbon groups and groups chosen from groups of formulae:

—(CH₂—CH₂—O)ₓ—CH₂—CH₂— and

—[CH₂—CH(CH₃)—O]_{y}—CH₂—CH(CH₃)—

wherein x and y, which are identical or different, are each chosen from integers ranging from 1 to 4 (in which case x and y represent a defined and unique degree of polymerization) and any number ranging from 1 to 4 (in which case x and y represent an average degree of polymerization),

b) bis-secondary diamine residues such as piperazine derivatives,

c) bis-primary diamine residues chosen from residues of formula: —NH—Y—NH—, wherein Y is chosen from linear and branched hydrocarbon groups and residues of formula

—CH₂—CH₂—S—S—CH₂—CH₂—, and

d) a ureylene group of formula: —NH—CO—NH—;

(3) quaternary diammonium polymers comprising repeating units of formula (IV):

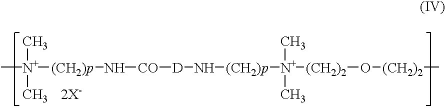

wherein:

p is an integer ranging from 1 to 6,

D is chosen from a direct bond and —(CH₂)ᵣ—CO— groups, wherein r is a number equal to 4 or 7, and

X⁻ is an anion;

(4) amine-containing silicones.

According to the present invention, in the broadest sense, said amine-containing silicones are cationic whether or not a quaternary ammonium group is present.

In the dyeing compositions according to the invention, the acid addition salts of said 1-(4-aminophenyl)pyrrolidines of formula (I) may be chosen, for example, from hydrochlorides, hydrobromides, sulphates, tartrates, lactates, and acetates.

The dyeing compositions in accordance with the invention, after combining with an oxidizing composition comprising at least one oxidizing agent, tend to produce colors that exhibit at least one of the following properties: (1) varied, chromatic, intense, and/or aesthetic shades, (2) low selectivity, (3) resistance to atmospheric agents such as light and adverse weather conditions, and (4) resistance to perspiration and various treatments to which the hair may be subjected.

Another subject of the invention relates to a ready-to-use composition for oxidation dyeing keratinous fibers comprising, in a medium suitable for dyeing:

(i) at least one oxidation dye precursor chosen from 1-(4-aminophenyl)-pyrrolidines of formula (I) and acid addition salts thereof:

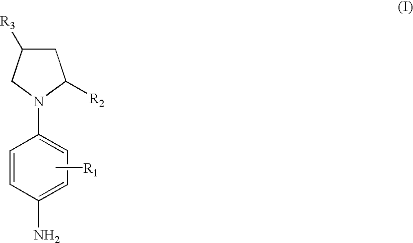

wherein:

R₁ is chosen from a hydrogen atom, C₁-C₆ alkyl groups, C₁-C₅ monohydroxyalkyl groups, and C₂-C₅ polyhydroxyalkyl groups,

R₂ is chosen from a hydrogen atom, a —CONH₂ group, C₁-C₅ monohydroxyalkyl groups, and C₂-C₅ polyhydroxyalkyl groups, and

R₃ is chosen from a hydrogen atom, and a hydroxyl group, and

(ii) at least one cationic polymer chosen from:

(1) homopolymers and copolymers comprising, as a constituent of the chain, at least one unit chosen from units formula (II):

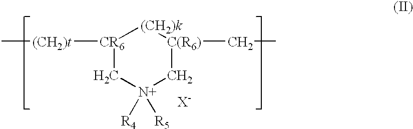

wherein:

k and t, which are identical or different, are each chosen from 0 and 1, provided that the sum of k+t is equal to 1,

R₄ and R₅, which are identical or different, are each chosen from (C₁-C₂₂) alkyl groups, (C₁-C₅)-hydroxyalkyl groups, and (C₁-C₄)amidoalkyl groups,

R₄ and R₅, together with the nitrogen cation to which they are commonly bonded, optionally form a cationic heterocyclic group chosen from a piperidinyl group and a morpholinyl group,

R₆, which are identical or different, are each chosen from a hydrogen atom and a methyl group, and

X⁻ is an anion;

(2) quaternary diammonium polymers comprising repeating units of formula (III):

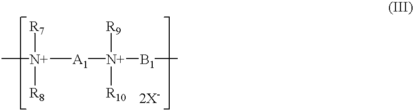

wherein:

R₇, R₈, R₉, and R₁₀, which are identical or different, are each chosen from C₁-C₂₀ aliphatic groups, C₃-C₂₀ alicyclic groups, C₇-C₂₀ arylaliphatic groups, and lower hydroxyalkyl groups,

at least two of said R₇, said R₈, said R₉, and said R₁₀, together with the nitrogen cations to which they are attached, optionally form at least one cationic heterocyclic ring optionally comprising an additional heteroatom other than nitrogen,

R₇, R₈, R₉, and R₁₀, which are identical or different, optionally are each chosen from linear and branched C₁-C₆ alkyl groups substituted with at least one group chosen from nitrile groups, ester groups, acyl groups, amide groups, —CO—O—R₁₁—D groups, and —CO—NH—R₁₁—D groups, wherein R₁₁ is chosen from alkylene groups and D is chosen from quaternary ammonium groups,

A₁ and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,

X⁻ is an anion,

said A₁, said R₇, and said R₉ optionally form a piperazine ring, together with the two nitrogen cations to which they are attached, and

provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is additionally chosen from —(CH₂)ₙ—CO—D—OC—(CH₂)ₙ— groups, wherein:

n is an integer ranging from 1 to 100, such as, for example, from 1 to 50, and

D is chosen from:

a) glycol residues of formula: —O—Z—O—, wherein Z is chosen from linear and branched hydrocarbon groups and groups chosen from groups of formulae:

—(CH₂—CH₂—O)ₓ—CH₂—CH₂— and

—[CH₂—CH(CH₃)—O]_{y}—CH₂—CH(CH₃)—

wherein x and y, which are identical or different, are each chosen from integers ranging from 1 to 4 (in which case x and y represent a defined and unique degree of polymerization) and any number ranging from 1 to 4 (in which case x and y represent an average degree of polymerization),

b) bis-secondary diamine residues such as piperazine derivatives,

c) bis-primary diamine residues chosen from residues of formula: —NH—Y—NH—, wherein Y is chosen from linear and branched hydrocarbon groups and residues of formula

—CH₂—CH₂—S—S—CH₂—CH₂—, and

d) a ureylene group of formula: —NH—CO—NH—;

(3) quaternary diammonium polymers comprising repeating units of formula (IV):

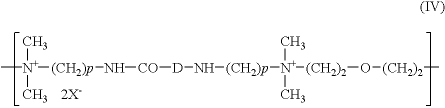

wherein:

p is an integer ranging from 1 to 6,

D is chosen from a direct bond and —(CH₂)ᵣ—CO— groups, wherein r is a number equal to 4 or 7, and

X⁻ is an anion;

(4) amine-containing silicones, and

(iii) at least one oxidizing agent.

The term “ready-to-use composition” is understood to mean, for the purposes of the present invention, a composition intended to be applied immediately to the keratinous fibers, either stored as it is before use or obtained from the combination of two or more compositions.

The invention also relates to a method for oxidation dyeing keratinous fibers, for example, human keratinous fibers such as hair, comprising:

(A) applying to said fibers at least one dyeing composition comprising, in a medium suitable for dyeing:

(i) at least one oxidation dye precursor chosen from 1-(4-aminophenyl)-pyrrolidines of formula (I) and acid addition salts thereof:

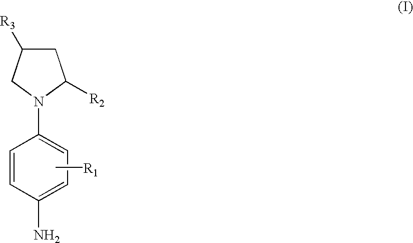

wherein:

R₁ is chosen from a hydrogen atom, C₁-C₆ alkyl groups, C₁-C₅ monohydroxyalkyl groups, and C₂-C₅ polyhydroxyalkyl groups,

R₂ is chosen from a hydrogen atom, a —CONH₂ group, C₁-C₅ monohydroxyalkyl groups, and C₂-C₅ polyhydroxyalkyl groups, and

R₃ is chosen from a hydrogen atom, and a hydroxyl group, and optionally comprising:

(ii) at least one cationic polymer chosen from:

(1) homopolymers and copolymers comprising, as a constituent of the chain, at least one unit chosen from units formula (II):

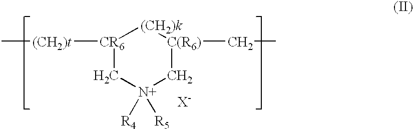

wherein:

k and t, which are identical or different, are each chosen from 0 and 1, provided that the sum of k+t is equal to 1,

R₄ and R₅, which are identical or different, are each chosen from (C₁-C₂₂) alkyl groups, (C₁-C₅)-hydroxyalkyl groups, and (C₁-C₄)amidoalkyl groups,

R₄ and R₅, together with the nitrogen cation to which they are commonly bonded, may optionally form a cationic heterocyclic group chosen from a piperidinyl group and a morpholinyl group,

R₆, which are identical or different, are each chosen from a hydrogen atom and a methyl group, and

X⁻ is an anion;

(2) quaternary diammonium polymers comprising repeating units of formula (III):

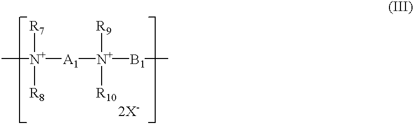

wherein:

R₇, R₈, R₉, and R₁₀, which are identical or different, are each chosen from C₁-C₂₀ aliphatic groups, C₃-C₂₀ alicyclic groups, C₇-C₂₀ arylaliphatic groups, and lower hydroxyalkyl groups,

at least two of said R₇, said R₈, said R₉, and said R₁₀, together with the nitrogen cations to which they are attached, optionally form at least one cationic heterocyclic ring optionally comprising an additional heteroatom other than nitrogen,

R₇, R₈, R₉, and R₁₀, which are identical or different, optionally are each chosen from linear and branched C₁-C₆ alkyl groups substituted with at least one group chosen from nitrile groups, ester groups, acyl groups, amide groups, —CO—O—R₁₁—D groups, and —CO—NH—R₁₁—D groups, wherein R₁₁ is chosen from alkylene groups and D is chosen from quaternary ammonium groups,

A₁ and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,

X⁻ is an anion,

said A₁, said R₇, and said R₉ optionally form a piperazine ring, together with the two nitrogen cations to which they are attached, and

provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is additionally chosen from

—(CH₂)ₙ—CO—D—OC—(CH₂)ₙ— groups, wherein:

n is an integer ranging from 1 to 100, such as, for example, from 1 to 50, and

D is chosen from:

a) glycol residues of formula: —O—Z—O—, wherein Z is chosen from linear and branched hydrocarbon groups and groups chosen from groups of formulae:

—(CH₂—CH₂—O)ₓ—CH₂—CH₂— and

—[CH₂—CH(CH₃)—O]_{y}—CH₂—CH(CH₃)—

wherein x and y, which are identical or different, are each chosen from integers ranging from 1 to 4 (in which case x and y represent a defined and unique degree of polymerization) and any number ranging from 1 to 4 (in which case x and y represent an average degree of polymerization),

b) bis-secondary diamine residues such as piperazine derivatives,

c) bis-primary diamine residues chosen from residues of formula: —NH—Y—NH—, wherein Y is chosen from linear and branched hydrocarbon groups and residues of formula

—CH₂—CH₂—S—S—CH₂—CH₂—, and

d) a ureylene group of formula: —NH—CO—NH—;

(3) quaternary diammonium polymers comprising repeating units of formula (IV):

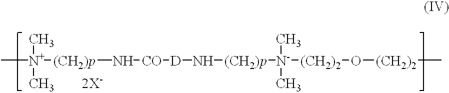

wherein:

p is an integer ranging from 1 to 6,

D is chosen from a direct bond and —(CH₂)ᵣ—CO— groups, wherein r is a number equal to 4 or 7, and

X⁻ is an anion;

(4) amine-containing silicones, and

(B) developing a color by applying to said fibers at least one oxidizing composition comprising:

at least one oxidizing agent and

optionally comprising said at least one cationic polymer as defined above,

wherein said at least one oxidizing composition is

applied to said fibers after combining, at the time of use, said at least one oxidizing composition with said at least one dyeing composition, or

applied to said fibers either simultaneously with or immediately after said at least one dyeing composition, without intermediate rinsing, and

(C) provided that said at least one cationic polymer is present in at least one of said at least one dyeing composition or said at least one oxidizing composition.

The invention also relates to a method for oxidation dyeing keratinous fibers, for example, human keratinous fibers such as hair, comprising:

(A) preparing, at the time of use, at least one dyeing composition comprising, in a medium suitable for dyeing:

(i) at least one oxidation dye precursor chosen from 1-(4-aminophenyl)-pyrrolidines of formula (I) and acid addition salts thereof:

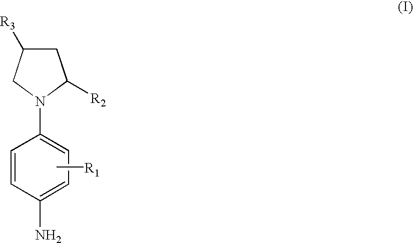

wherein:

R₁ is chosen from a hydrogen atom, C₁-C₆ alkyl groups, C₁-C₅ monohydroxyalkyl groups, and C₂-C₅ polyhydroxyalkyl groups,

R₂ is chosen from a hydrogen atom, a —CONH₂ group, C₁-C₅ monohydroxyalkyl groups, and C₂-C₅ polyhydroxyalkyl groups, and

R₃ is chosen from a hydrogen atom, and a hydroxyl group,

(ii) at least one cationic polymer chosen from:

(1) homopolymers and copolymers comprising, as a constituent of the chain, at least one unit chosen from units formula (II):

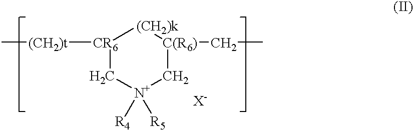

wherein:

k and t, which are identical or different, are each chosen from 0 and 1, provided that the sum of k+t is equal to 1,

R₄ and R₅, which are identical or different, are each chosen from (C₁-C₂₂) alkyl groups, (C₁-C₅)-hydroxyalkyl groups, and (C₁-C₄)amidoalkyl groups,

R₄ and R₅, together with the nitrogen cation to which they are commonly bonded, may optionally form a cationic heterocyclic group chosen from a piperidinyl group and a morpholinyl group,

R₆, which are identical or different, are each chosen from a hydrogen atom and a methyl group, and

X⁻ is an anion;

(2) quaternary diammonium polymers comprising repeating units of formula (III):

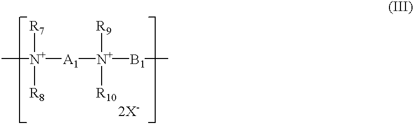

wherein:

R₇, R₈, R₉, and R₁₀, which are identical or different, are each chosen from C₁-C₂₀ aliphatic groups, C₃-C₂₀ alicyclic groups, C₇-C₂₀ arylaliphatic groups, and lower hydroxyalkyl groups,

at least two of said R₇, said R₈, said R₉, and said R₁₀, together with the nitrogen cations to which they are attached, optionally form at least one cationic heterocyclic ring optionally comprising an additional heteroatom other than nitrogen,

R₇, R₈, R₉, and R₁₀, which are identical or different, optionally are each chosen from linear and branched C₁-C₆ alkyl groups substituted with at least one group chosen from nitrile groups, ester groups, acyl groups, amide groups, —CO—O—R₁₁—D groups, and —CO—NH—R₁₁—D groups, wherein R₁₁ is chosen from alkylene groups and D is chosen from quaternary ammonium groups,

A₁ and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,

X⁻ is an anion,

said A₁, said R₇, and said R₉ optionally form a piperazine ring, together with the two nitrogen cations to which they are attached, and

provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is additionally chosen from

—(CH₂)ₙ—CO—D—OC—(CH₂)ₙ— groups, wherein:

n is an integer ranging from 1 to 100, such as, for example, from 1 to 50, and

D is chosen from:

a) glycol residues of formula: —O—Z—O—, wherein Z is chosen from linear and branched hydrocarbon groups and groups chosen from groups of formulae:

—(CH₂—CH₂—O)ₓ—CH₂—CH₂— and

—[CH₂—CH(CH₃)—O]_{y}—CH₂—CH(CH₃)—

wherein x and y, which are identical or different, are each chosen from integers ranging from 1 to 4 (in which case x and y represent a defined and unique degree of polymerization) and any number ranging from 1 to 4 (in which case x and y represent an average degree of polymerization),

b) bis-secondary diamine residues such as piperazine derivatives,

c) bis-primary diamine residues chosen from residues of formula: —NH—Y—NH—, wherein Y is chosen from linear and branched hydrocarbon groups and residues of formula

—CH₂—CH₂—S—S—CH₂—CH₂—, and

d) a ureylene group of formula: —NH—CO—NH—;

(3) quaternary diammonium polymers comprising repeating units of formula (IV):

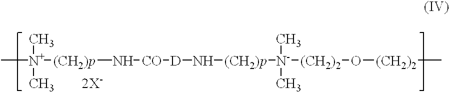

wherein:

p is an integer ranging from 1 to 6,

D is chosen from a direct bond and —(CH₂)ᵣ—CO— groups, wherein r is a number equal to 4 or 7, and

X⁻ is an anion;

(4) amine-containing silicones, and

(iii) at least one oxidizing agent,

wherein said at least one dyeing composition is prepared, at the time of use, by combining (i), (ii), and (iii) above,

(B) developing a color by applying said at least one dyeing composition prepared in (A) above to said fibers,

(C) leaving said at least one dyeing composition prepared in (A) above on said fibers for a time ranging, for example, from 1 to 60 minutes, such as, for example, from 10 to 45 minutes,

(D) rinsing said fibers, optionally shampooing said fibers, and optionally further rinsing said fibers, and

(E) drying said fibers.

According to the present invention, the hair that can be dyed in accordance with the method above can be chosen from, for example, wet hair and dry hair.

One embodiment of the invention relates to multicompartment dyeing devices or “kits” for oxidation dyeing keratinous fibers, for example, human keratinous fibers such as hair.

A kit for oxidation dyeing keratinous fibers according to the invention comprises at least two compartments, wherein:

(A) a first compartment comprises at least one dyeing composition comprising, in a medium suitable for dyeing:

(i) at least one oxidation dye precursor chosen from 1-(4-aminophenyl)-pyrrolidines of formula (I) and acid addition salts thereof:

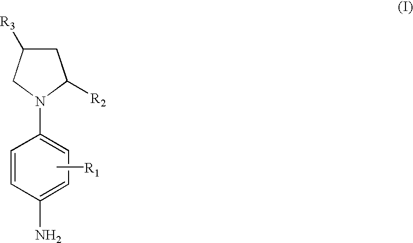

wherein:

R₁ is chosen from a hydrogen atom, C₁-C₆ alkyl groups, C₁-C₅ monohydroxyalkyl groups, and C₂-C₅ polyhydroxyalkyl groups,

R₂ is chosen from a hydrogen atom, a —CONH₂ group, C₁-C₅ monohydroxyalkyl groups, and C₂-C₅ polyhydroxyalkyl groups, and

R₃ is chosen from a hydrogen atom, and a hydroxyl group, and optionally comprising:

(ii) at least one cationic polymer chosen from:

(1) homopolymers and copolymers comprising, as a constituent of the chain, at least one unit chosen from units formula (II):

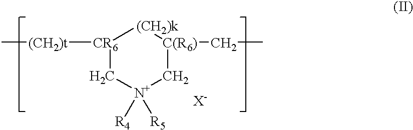

wherein:

k and t, which are identical or different, are each chosen from 0 and 1, provided that the sum of k+t is equal to 1,

R₄ and R₅, which are identical or different, are each chosen from (C₁-C₂₂) alkyl groups, (C₁-C₅)-hydroxyalkyl groups, and (C₁-C₄)amidoalkyl groups,

R₄ and R₅, together with the nitrogen cation to which they are commonly bonded, may optionally form a cationic heterocyclic group chosen from a piperidinyl group and a morpholinyl group,

R₆, which are identical or different, are each chosen from a hydrogen atom and a methyl group, and

X⁻ is an anion;

(2) quaternary diammonium polymers comprising repeating units of formula (III):

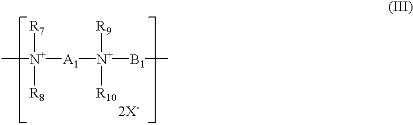

wherein:

R₇, R₈, R₉, and R₁₀, which are identical or different, are each chosen from C₁-C₂₀ aliphatic groups, C₃-C₂₀ alicyclic groups, C₇-C₂₀ arylaliphatic groups, and lower hydroxyalkyl groups,

at least two of said R₇, said R₈, said R₉, and said R₁₀, together with the nitrogen cations to which they are attached, optionally form at least one cationic heterocyclic ring optionally comprising an additional heteroatom other than nitrogen,

R₇, R₈, R₉, and R₁₀, which are identical or different, optionally are each chosen from linear and branched C₁-C₆ alkyl groups substituted with at least one group chosen from nitrile groups, ester groups, acyl groups, amide groups, —CO—O—R₁₁—D groups, and —CO—NH—R₁₁—D groups, wherein R₁₁ is chosen from alkylene groups and D is chosen from quaternary ammonium groups,

A₁ and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,

X⁻ is an anion,

said A₁, said R₇, and said R₉ optionally form a piperazine ring, together with the two nitrogen cations to which they are attached, and

provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is additionally chosen from

—(CH₂)ₙ—CO—D—OC—(CH₂)ₙ— groups, wherein:

n is an integer ranging from 1 to 100, such as, for example, from 1 to 50, and

D is chosen from:

a) glycol residues of formula: —O—Z—O—, wherein Z is chosen from linear and branched hydrocarbon groups and groups chosen from groups of formulae:

—(CH₂—CH₂—O)ₓ—CH₂—CH₂— and

—[CH₂—CH(CH₃)—O]_{y}—CH₂—CH(CH₃)—

wherein x and y, which are identical or different, are each chosen from integers ranging from 1 to 4 (in which case x and y represent a defined and unique degree of polymerization) and any number ranging from 1 to 4 (in which case x and y represent an average degree of polymerization),

b) bis-secondary diamine residues such as piperazine derivatives,

c) bis-primary diamine residues chosen from residues of formula: —NH—Y—NH—, wherein Y is chosen from linear and branched hydrocarbon groups and residues of formula

—CH₂—CH₂—S—S—CH₂—CH₂—, and

d) a ureylene group of formula: —NH—CO—NH—;

(3) quaternary diammonium polymers comprising repeating units of formula (IV):

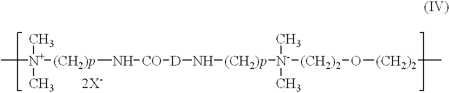

wherein:

p is an integer ranging from 1 to 6,

D is chosen from a direct bond and —(CH₂)ᵣ—CO— groups, wherein r is a number equal to 4 or 7, and

X⁻ is an anion; and

(4) amine-containing silicones, and

(B) a second compartment comprises at least one oxidizing agent and optionally comprises said at least one cationic polymer as defined above, and

(C) provided that said at least one cationic polymer is present in at least one of said first compartment or said second compartment.

Another aspect of the invention relates to a kit for oxidation dyeing keratinous fibers comprising at least three compartments, wherein:

(A) a first compartment comprises at least one dyeing composition comprising, in a medium suitable for dyeing, at least one oxidation dye precursor chosen from 1-(4-aminophenyl)-pyrrolidines of formula (I) and acid addition salts thereof:

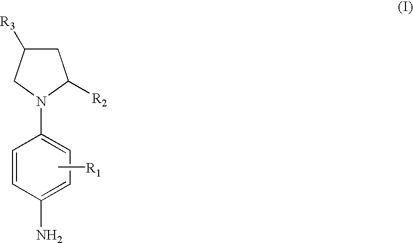

wherein:

R₁ is chosen from a hydrogen atom, C₁-C₆ alkyl groups, C₁-C₅ monohydroxyalkyl groups, and C₂-C₅ polyhydroxyalkyl groups,

R₂ is chosen from a hydrogen atom, a —CONH₂ group, C₁-C₅ monohydroxyalkyl groups, and C₂-C₅ polyhydroxyalkyl groups, and

R₃ is chosen from a hydrogen atom, and a hydroxyl group,

(B) a second compartment comprises at least one cationic polymer chosen from:

(1) homopolymers and copolymers comprising, as a constituent of the chain, at least one unit chosen from units formula (II):

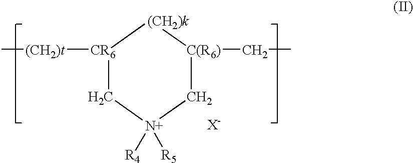

wherein:

k and t, which are identical or different, are each chosen from 0 and 1, with the proviso that the sum of k+t is equal to 1,

R₄ and R₅, which are identical or different, are each chosen from (C₁-C₂₂) alkyl groups, (C₁-C₅)-hydroxyalkyl groups, and (C₁-C₄)amidoalkyl groups,

R₄ and R₅, together with the nitrogen cation to which they are commonly bonded, may optionally form a cationic heterocyclic group chosen from a piperidinyl group and a morpholinyl group,

R₆, which are identical or different, are each chosen from a hydrogen atom and a methyl group, and

X⁻ is an anion;

(2) quaternary diammonium polymers comprising repeating units of formula (III):

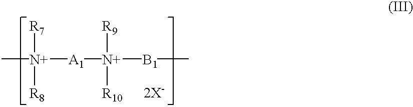

wherein:

R₇, R₈, R₉, and R₁₀, which are identical or different, are each chosen from C₁-C₂₀ aliphatic groups, C₃-C₂₀ alicyclic groups, C₇-C₂₀ arylaliphatic groups, and lower hydroxyalkyl groups,

at least two of said R₇, said R₈, said R₉, and said R₁₀, together with the nitrogen cations to which they are attached, optionally form at least one cationic heterocyclic ring optionally comprising an additional heteroatom other than nitrogen,

R₇, R₈, R₉, and R₁₀, which are identical or different, optionally are each chosen from linear and branched C₁-C₆ alkyl groups substituted with at least one group chosen from nitrile groups, ester groups, acyl groups, amide groups, —CO—O—R₁₁—D groups, and —CO—NH—R₁₁—D groups, wherein R₁₁ is chosen from alkylene groups and D is chosen from quaternary ammonium groups,

A₁ and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,

X⁻ is an anion,

said A₁, said R₇, and said R₉ optionally form a piperazine ring, together with the two nitrogen cations to which they are attached, and

provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is additionally chosen from

—(CH₂)ₙ—CO—D—OC—(CH₂)ₙ— groups, wherein:

n is an integer ranging from 1 to 100, such as, for example, from 1 to 50, and

D is chosen from:

a) glycol residues of formula: —O—Z—O—, wherein Z is chosen from linear and branched hydrocarbon groups and groups chosen from groups of formulae:

—(CH₂—CH₂—O)ₓ—CH₂—CH₂— and

—[CH₂—CH(CH₃)—O]_{y}—CH₂—CH(CH₃)—

wherein x and y, which are identical or different, are each chosen from integers ranging from 1 to 4 (in which case x and y represent a defined and unique degree of polymerization) and any number ranging from 1 to 4 (in which case x and y represent an average degree of polymerization),

b) bis-secondary diamine residues such as piperazine derivatives,

c) bis-primary diamine residues chosen from residues of formula: —NH—Y—NH—, wherein Y is chosen from linear and branched hydrocarbon groups and residues of formula

—CH₂—CH₂—S—S—CH₂—CH₂—, and

d) a ureylene group of formula: —NH—CO—NH—;

(3) quaternary diammonium polymers comprising repeating units of formula (IV):

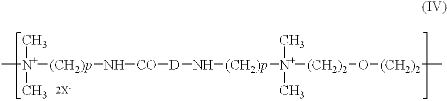

wherein:

p is an integer ranging from 1 to 6,

D is chosen from a direct bond and —(CH₂)ᵣ—CO— groups, wherein r is a number equal to 4 or 7, and

X⁻ is an anion; and

(4) amine-containing silicones, and

(C) a third compartment comprises at least one oxidizing agent.

Additional characteristics, aspects, objects, and advantages of the invention will emerge even more clearly on reading the description and examples which follow without however exhibiting a limiting character.

Representative 1-(4-aminophenyl)pyrrolidines of formula (I) according to the invention are, for example, described and prepared in U.S. Pat. Nos. 5,851,237, 5,876,464, and 5,993,491, the disclosures of which are incorporated by reference herein.

Representative 1-(4-aminophenyl)-pyrrolidines of formula (I) that can be used according to the invention can be chosen from such compounds wherein:

R₁, R₂, and R₃ are each a hydrogen atom, wherein said compound of formula (I) is 1-(4-aminophenyl)pyrrolidine,

R₁ and R₃ are each a hydrogen atom and R₂ is a —CH₂OH group, wherein said compound of formula (I) is 1-(4-aminophenyl)-2-pyrrolidinemethanol,

R₁ is a hydrogen atom, R₂ is a —CH₂OH group and R₃ is a hydroxyl group, wherein said compound of formula (I) is 1-(4-aminophenyl)-4-hydroxy-2-pyrrolidinemethanol, and

R₁ and R₃ are each a hydrogen atom and R₂ is a —CONH₂ group, wherein said compound of formula (I) is N-(4-aminophenyl)prolineamide.

The 1-(4-aminophenyl)pyrrolidines of formula (I) and acid addition salts thereof used in accordance with the invention can be present in the composition in an amount ranging, for example, from 0.001% to 10% by weight relative to the total weight of the composition, such as, for example, from 0.01% to 8% by weight relative to the total weight of the composition.

The cationic polymers which can be used according to the invention generally have a number-average molecular mass ranging, for example, from 500 to 5×10⁶, such as, for example, ranging from 1×10³ to 3×10⁶.

Representatives of the at least one cationic polymer that can be used according to the invention can include said cationic polymers comprising repeating units of formula (II), wherein said R₄ and said R₅, which are identical or different, are each chosen from (C₁-C₄) alkyl groups, X⁻ is an anion chosen from a bromide anion, a chloride anion, an acetate anion, a borate anion, a citrate anion, a tartrate anion, a bisulphate anion, a bisulphite anion, a sulphate anion, and a phosphate anion. Such polymers, for example, are described in French Patent 2,080,759 and in its certificate of addition 2,190,406, the disclosures of which are incorporated by reference herein.

The cationic polymers comprising repeating units of formula (II) can include, for example, the homopolymer of dimethyldiallylammonium chloride sold under the name “Merquat 100” by the company Calgon (and its homologues of low weight-average molecular mass) and the copolymers of dimethyldiallylammonium chloride and of acrylamide marketed under the name “MERQUAT 550”.

The cationic polymers comprising repeating units of formula (III) that can be used according to the invention, for example, can have a number-average molecular mass generally ranging, for example, from 1,000 to 100,000 and are cationic polymers, wherein X⁻ is an anion chosen from a chloride anion and a bromide anion.

For example, the cationic polymers comprising repeating units of formula (III) are described, for example, in French Patents 2,320,330, 2,270,846, 2,316,271, 2,336,434, and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020, the disclosures of which are incorporated by reference herein.

Further, representative quaternary diammonium polymers comprising repeating units of formula (III) can, for example, comprise repeating units of formula (V):

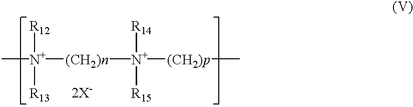

wherein

R₁₂, R₁₃, R₁₄, and R₁₅, which are identical or different, are each chosen from C₁-C₄ alkyl groups and C₁-C₄ hydroxyalkyl groups,

n and p are each chosen from integers ranging from 2 to 20, and

X⁻ is an anion.

Such cationic polymers can be chosen from, for example, cationic polymers comprising repeating units of formula (V), wherein R₁₂, R₁₃, R₁₄, and R₁₅, which are identical or different, are each chosen from a methyl group and an ethyl group.

Cationic polymers comprising repeating units of formula (V) can include, for example, those for which R₁₂, R₁₃, R₁₄, and R₁₅ are each a methyl group and n=3, p=6 and X⁻=Cl⁻ and, for example, cationic polymers having a number-average molecular weight, determined by gel permeation chromatography, ranging from, for example, 9,500 to 9,900. Such cationic polymers are cationic polymers comprising repeating units (W):

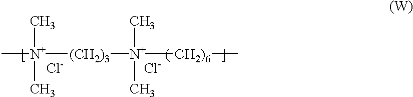

Cationic polymers comprising repeating units of formula (V) can include, for example, those for which R₁₂ and R₁₃ are each a methyl group, R₁₄ and R₁₅ are each an ethyl group, n=p=3, and X⁻=Br⁻ and further, such as, for example, cationic polymers having a number-average molecular weight, determined by gel permeation chromatography, for example, of about 1,200. Such cationic polymers are cationic polymers comprising repeating units (U):

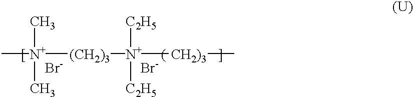

Said cationic polymers with (W) repeating units and said cationic polymers with (U) repeating units are prepared and described, for example, in French Patent 2,270,846, the disclosure of which is incorporated by reference herein.

Said cationic polymers comprising repeating units of formula (IV) are, for example, described in Patent Application EP-A-122 324, the disclosure of which is incorporated by reference herein.

Representatives of said cationic polymers comprising repeating units of formula (IV) can include, for example, cationic polymers having a number-average molecular mass, measured by Carbon 13 NMR, of less than 100,000, and wherein, p is chosen from integers ranging from 1 to 6, D is chosen from a direct bond and —(CH₂)ᵣ—CO— groups, wherein r is a number equal to 4 or 7, and X⁻ is an anion chosen from anions derived from an inorganic acid and anions derived from an organic acid.

In one embodiment of the invention, said cationic polymers comprising repeating units of formula (IV) are chosen from:

a) repeating units of formula (IV), wherein p is equal to 3, D is a —(CH₂)₄—CO— group, X⁻ is a chloride anion, and the number-average molecular mass, measured by Carbon 13 NMR (¹³C NMR) is 5,600; a polymer of this type is provided by the company MIRANOL under the name MIRAPOL-AD1,

b) repeating units of formula (IV), wherein p is equal to 3, D is a —(CH₂)₇—CO— group, X⁻ is a chloride anion, and the number-average molecular mass, measured by Carbon 13 NMR (¹³C NMR) is 8,100; a polymer of this type is provided by the company MIRANOL under the name MIRAPOL-AZ1,

c) repeating units of formula (IV), wherein p is equal to 3, D is a direct bond, X⁻ is a chloride anion, and the number-average molecular mass, measured by Carbon 13 NMR (¹³C NMR) is 25,500; a polymer of this type is sold by the company MIRANOL under the name MIRAPOL-A15, and

d) repeating units of formula (IV) described in sections a) and c) above, wherein a “Block Copolymer” is formed; polymers of this type include polymers provided by the company MIRANOL, under the names MIRAPOL-9, (wherein the number-average molecular mass, measured by Carbon 13 NMR (¹³C NMR) is 7,800) MIRAPOL-175, (wherein the number-average molecular mass, measured by Carbon 13 NMR (¹³C NMR) is 8,000), and MIRAPOL-95 (wherein the number-average molecular mass, measured by Carbon 13 NMR (¹³C NMR) is 12,500).

Further, for example, according to the invention, said cationic polymers comprising repeating units of formula (IV) wherein p is equal to 3, D is a direct bond, X⁻ is a chloride ion, and the number-average molecular mass, measured by Carbon 13 NMR (¹³C NMR) is 25,500 can also be used.

Said representative cationic polymers comprising repeating units of formula (IV) may be prepared, for example, according to the methods described in U.S. Pat. Nos. 4,157,388, 4,390,689, 4,702,906, and 4,719,282, the disclosures of which are incorporated by reference herein.

According to the invention, said at least one amine-containing silicone is intended to mean any silicone comprising at least one primary, secondary or tertiary amine or a quaternary ammonium group. Further, in the broadest sense, said at least one amine-containing silicone is cationic whether or not a quaternary ammonium group is present.

In accordance with the invention, said at least one amine-containing silicone is chosen from:

(i) polysiloxanes referred to in the CTFA dictionary as “amodimethicone” of formula (VI):

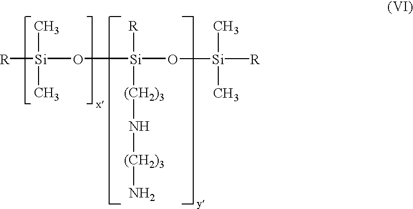

wherein:

R is a group chosen from a methyl group and a hydroxyl group, and

x′ and y′ are integers chosen such that generally the weight-average molecular weight of said polysiloxane ranges from 5,000 to 500,000;

(ii) aminosilicones of formula (VII):

(R¹)ₐ(T)₃₋ₐ-Si[OSi(T)₂]ₙ-[OSi(T)_{b}(R¹)_{2-b})]ₘ—O—Si(T)₃₋ₐ—(R¹)ₐ (VII)

wherein:

T is chosen from a hydrogen atom, a phenyl group, a hydroxyl group, and (C₁-C₈) alkyl groups, for example a methyl group,

a is an integer ranging from 0 to 3, and in one embodiment a is 0,

b is chosen from 0 and 1, and in one embodiment b is 1,

m and n are numbers such that the sum (n+m) ranges, for example, from 1 to 2,000, such as, for example, from 50 to 150, and wherein n can be chosen from a number ranging, for example, from 0 to 1,999, such as, for example, from 49 to 149, and wherein m can be chosen from a number ranging, for example, from 1 to 2,000, such as for example from 1 to 10;

R¹ is a monovalent group of formula —C_{q}H_{2q}L, wherein q is chosen from a number ranging, for example, from 2 to 8, and wherein L is an optionally quaternized amine group chosen from:

—N(R²)—CH₂—CH₂—N(R²)₂,

—N(R²)₂,

—N⁺(R²)₃Q⁻,

—N⁺(R²)(H)₂Q⁻,

—N⁺(R²)₂HQ⁻, and

—N(R²)—CH₂—CH₂—N⁺(R²)H₂Q⁻,

in which R², which are identical or different, can each be chosen from a hydrogen atom, a phenyl group, a benzyl group, and saturated monovalent hydrocarbon-based groups, such as, for example, (C₁-C₂₀) alkyl groups, and Q⁻ is chosen from halide anions such as, for example, a fluoride anion, a chloride anion, a bromide anion, and an iodide anion.

In one embodiment said aminosilicones of formula (VII) can be chosen from “trimethylsilylamodimethicones” of formula (VIII):

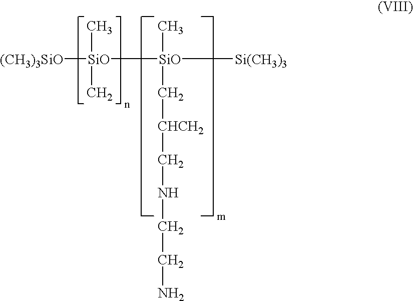

wherein:

m and n are numbers such that the sum (n+m) ranges, for example, from 1 to 2,000, such as, for example, from 50 to 150, and wherein n can be chosen from a number ranging, for example, from 0 to 1,999, such as, for example, from 49 to 149, and wherein m can be chosen from a number ranging, for example, from 1 to 2,000, such as for example from 1 to 10.

Such polymers are described, for example, in patent application EP-A-95238, the disclosure of which is incorporated herein by reference. A compound of formula (VIII) is sold, for example, under the name Q2-8220 by the company OSI.

Further, in accordance with the invention, said at least one amine-containing silicone is chosen from:

(iii) aminosilicones of formula (IX):

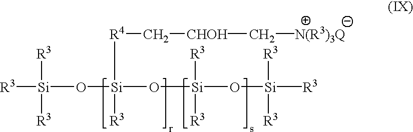

wherein:

R³, which are identical or different, are each chosen from (C₁-C₁₈) monovalent hydrocarbon-based groups, such as, for example, (C₁-C₁₈) alkyl groups and (C₂-C₁₈) alkenyl groups,

R₄ is chosen from divalent hydrocarbon-based groups, such as, for example, divalent (C₁-C₁₈) alkylene groups, and divalent (C₁-C₁₈) alkyleneoxy groups, such as, for example, (C₁-C₈) alkyleneoxy groups,

Q⁻ is chosen from halide anions, such as, for example, a chloride anion,

r is a mean statistical value ranging from 2 to 20, such as, for example, from 2 to 8, and

s is a mean statistical value ranging from 20 to 200, such as, for example, from 20 to 50.

Such aminosilicones are described, for example, in U.S. Pat. No. 4,185,087, the disclosure of which is incorporated by reference herein. An aminosilicone that falls within this class is the aminosilicone sold by the company Union Carbide under the name “Ucar Silicone ALE 56”.

When said composition according to the invention comprises at least one amine-containing silicone chosen from the amine-containing silicones of formulae (VI), (VII), (VIII), and (IX), said composition can, for example, comprise at least one surfactant chosen from, for example, cationic surfactants and nonionic surfactants.

By way of example, there may be mentioned the product sold under the name “Emulsion Cationic DC 939” by the company Dow Corning, which comprises, in addition to amodimethicone, a cationic surfactant, trimethylcetylammonium chloride, and a nonionic surfactant of formula: C₁₃H₂₇—(OC₂H₄)₁₂—OH, known under the CTFA name “trideceth-12”.

Another commercial product which can be used according to the invention is the product sold under the name “DOW CORNING Q2 7224” by the company Dow Corning, comprising, in combination, the trimethylsilylamodimethicone of formula (VIII) described above, a nonionic surfactant of formula: C₈H₁₇—C₆H₄—(OCH₂CH₂)₄₀—OH, known by the CTFA name “octoxynol-40”, a second nonionic surfactant of formula: C₁₂H₂₅—(OCH₂—CH₂)₆—OH, known under the CTFA name “isolaureth-6”, and propylene glycol.

Further, in accordance with the invention, said at least one amine-containing silicone is chosen from:

(iv) silicones of formula (X):

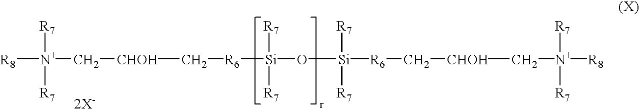

wherein:

R₆ is chosen from divalent hydrocarbon-based groups, such as, for example, (C₁-C₁₈) alkylene groups, and from divalent (C₁-C₁₈) alkyleneoxy groups, wherein said R₆ is bonded to the Si by way of an SiC bond,

R₇, which are identical or different, are each chosen from (C₁-C₁₈) monovalent hydrocarbon-based groups, (C₂-C₁₈) alkenyl groups, and (C₅-C₆) rings,

R₈, which are identical or different, are each chosen from a hydrogen atom, (C₁-C₁₈) monovalent hydrocarbon-based groups, (C₂-C₁₈) alkenyl groups, and —R₆—NHCOR₇ groups, wherein said R₆ and said R₇ are defined above,

r is a mean statistical value ranging from 2 to 200, and

X⁻ is an anion chosen, for example, from halides, such as, for example, a chloride anion, and from organic acid salts, such as, for example, acetate.

The silicones of formula (X) are, for example, described in application EP-A-0530974, the disclosure of which is incorporated by reference herein. Silicones of this class are, for example, the silicones marketed by the company Goldschmidt under the names: ABIL QUAT 3270, ABIL QUAT 3272, and ABIL QUAT 3474.

The at least one cationic polymer can be present in the composition according to the invention in an amount ranging from, for example, 0.01% to 10% by weight relative to the total weight of the composition, such as, for example, from 0.05% to 5% by weight relative to the total weight of the composition, and further such as, for example, from 0.1% to 3% by weight relative to the total weight of the composition.

The compositions according to the invention can also comprise at least one coupler. Representatives of the at least one coupler can include, for example, meta-phenylenediamines, meta-aminophenols, meta-diphenols, naphthols, and heterocyclic couplers, such as, for example, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, and acid addition salts of any of the foregoing compounds.

Representatives of the at least one coupler can include, for example, couplers chosen from 2,4-diamino-1-(β-hydroxyethyloxy)benzene, 2-methyl-5-aminophenol, 5-N-(β-hydroxyethyl) amino-2-methylphenol, 3-aminophenol, 1,3-dihydroxybenzene, 1,3-dihydroxy-2-methylbenzene, 4-chloro-1,3-dihydroxy-benzene, 2-amino-4-(β-hydroxyethylamino)-1-methoxy-benzene, 1,3-diaminobenzene, 1,3-bis(2,4-diamino-phenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxy-indoline, 2,6-dihydroxy-4-methylpyridine, 1-H-3-methyl-pyrazol-5-one, 1-phenyl-3-methylpyrazol-5-one, 2-amino-3-hydroxypyridine, 3,6-dimethylpyrazolo[3,2-c]-1,2,4-triazole, 2,6-dimethylpyrazolo[1,5-b]-1,2,4-triazole and acid addition salts of any of the foregoing compounds.

The at least one coupler can be present in the composition according to the invention in an amount ranging, for example, from 0.0001% to 15% by weight relative to the total weight of the composition.

The dyeing compositions in accordance with the invention may, in addition, comprise at least one direct dye. Further, the dyeing compositions in accordance with the invention may, in addition, comprise at least one additional oxidation base chosen from oxidation bases other than said 1-(4-aminophenyl)pyrrolidines of formula (I) and acid addition salts thereof.

Representatives of the at least one additional oxidation bases which can be used according to the invention, include, for example, para-phenylenediamine, para- tolylenediamine, 2-hydroxyethyl-para-phenylenediamine, 1-N,N-bis(2-hydroxyethyl)-para-phenylenediamine, para-aminophenols such as 3-methyl-4-aminophenol and 4-aminophenol, ortho-phenylenediamines, ortho-aminophenols, double bases, and heterocyclic bases, such as pyrimidines, for example, 2,4,5,6-tetraaminopyrimidine, and pyrazoles, for example, 1-(2-hydroxyethyl)-4,5-diaminopyrazole, and acid addition salts of any of the foregoing compounds.

Said at least one additional oxidation base may be present in the composition according to the invention in an amount ranging, for example, from 0.0001% to 15% by weight relative to the total weight of said composition.

One embodiment of the dyeing composition in accordance with the invention may, in addition, comprise at least one direct dye, for example, to modify the shades of the dyes by enriching them with glints. The at least one direct dye may be chosen from neutral, cationic, and anionic nitro dyes; neutral, cationic, and anionic azo dyes; and neutral, cationic, and anionic anthraquinone dyes.

The at least one direct dye can be present in the composition according to the invention in an amount ranging, for example, from 0.001% to 20% by weight relative to the total weight of said composition, such as, for example, from 0.01% to 10% by weight relative to the total weight of the composition.

The medium suitable for dyeing according to the invention can be, for example, an aqueous medium comprising water and may further comprise at least one cosmetically acceptable organic solvent. The at least one cosmetically acceptable organic solvent may, for example, be chosen from alcohols, such as ethyl alcohol, isopropyl alcohol, benzyl alcohol, and phenylethyl alcohol; glycols (for example, ethyleneglycol, propyleneglycol, butyleneglycol, dipropyleneglycol, and diethyleneglycol); and ethers of glycols (for example, monomethyl, monoethyl and monobutyl ethers of ethyleneglycol and for example monomethyl ether of propyleneglycol and alkyl ethers of diethyleneglycol glycol, such as, for example, monoethylether and monobutylether of diethyleneglycol). The at least one cosmetically acceptable organic solvent may be present in the composition according to the invention in an amount ranging from, for example, 0.5% to 20% by weight relative to the total weight of the composition, such as, for example, from 2% to 10% by weight relative to the total weight of the composition.

The composition according to the invention may further comprise an effective quantity of other agents. For example, agents that are already known in oxidation dyeing, such as various customary adjuvants such as sequestrants such as EDTA and etidronic acid, UV-screening agents, waxes, volatile and nonvolatile, cyclic and linear, branched and unbranched silicones, preservatives, ceramides, pseudoceramides, vegetable, mineral, and synthetic oils, vitamins and provitamins such as panthenol, opacifiers, thickening agents such as crosslinked polyacrylic acids and hydroxyalkyl celluloses, may be added.

The compositions may also comprise at least one agent chosen from reducing agents and antioxidants. Representative agents may include, for example, sodium sulphite, thioglycolic acid, thiolactic acid, sodium bisulphite, dehydroascorbic acid, hydroquinone, 2-methylhydroquinone, tert-butylhydroquinone, and homogentisic acid. Such agents are generally present in the composition of the invention in an amount ranging from, for example, 0.05% to 1.5% by weight relative to the total weight of the composition.

The composition according to the invention may also comprise at least one fatty alcohol. Representatives of the at least one fatty alcohol that can be used according to the invention can include, for example, lauryl alcohol, cetyl alcohol, stearyl alcohol, and oleyl alcohol. The at least one fatty alcohol may be present in the composition according to the invention in an amount ranging, for example, from 0.001% to 20% by weight relative to the total weight of the composition.

The compositions according to the invention may further comprise at least one surfactant chosen from nonionic, anionic, cationic, and amphoteric surfactants. Said at least one surfactant may be present in the composition according to the invention in at least one of said at least one dyeing composition or said at least one oxidizing composition. Said at least one surfactant can be present in the composition according to the invention an amount ranging, for example, from 0.1% to 20% by weight relative to the total weight of said composition.

In one embodiment, the composition according to the invention comprises at least one nonionic surfactant.

One skilled in the art should take care to select said optionally complementary compounds, such that the advantageous properties intrinsically associated with the dye composition according to the invention are not, or are not substantially, adversely affected by the additions envisaged.

Said at least one oxidizing composition according to the invention comprises at least one oxidizing agent. Said at least one oxidizing agent can, for example, be chosen from hydrogen peroxide, urea peroxide, alkali metal bromates, alkali metal ferricyanides, and persalts such as perborates and persulphates. In one embodiment, said at least one oxidizing agent can, for example, be hydrogen peroxide. When said at least one oxidizing agent is hydrogen peroxide, said at least one oxidizing agent may, for example, comprise a solution of hydrogen peroxide with a titre ranging, for example, from 1 to 40 in volume, such as, for example, from 5 to 40 in volume.

It is also possible to use as said at least one oxidizing agent at least one oxidation-reduction enzyme. Said oxidation-reduction enzymes can be chosen from, for example, laccases, peroxidases, and 2-electron oxidoreductases (such as uricase), where appropriate in the presence of their respective donor or cofactor.

The pH of the dyeing composition or of the ready-to-use composition (composition resulting from combining said at least one dyeing composition with said at least one oxidizing composition) applied to the keratinous fibers, generally ranges, for example, from 3 to 12, such as, for example, from 6 to 11. Said pH may be adjusted to the desired value by means of at least one agent for adjusting said pH chosen from acidifying agents and alkalinizing agents well known in the state of the art for dyeing keratinous fibers.

Representative alkalinizing agents may be chosen from, for example, aqueous ammonia, alkali metal carbonates, alkanolamines such as mono-, di- and triethanolamines as well their derivatives, oxyethylenated ethylenediamines, oxypropylenated ethylenediamines, hydroxyalkylamines, sodium hydroxide, potassium hydroxide, and compounds of formula (XI):

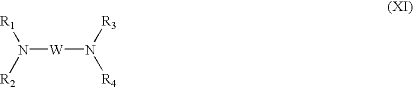

wherein:

W is a propylene residue optionally substituted with a group chosen from a hydroxyl group and C₁-C₄ alkyl groups;

R₁, R₂, R₃, and R₄, which are identical or different, are each chosen from hydrogen, C₁-C₄ alkyl groups, and C₁-C₄ hydroxyalkyl groups.

The acidifying agents are conventionally, by way of example, chosen from, inorganic acids, and organic acids, such as, for example, hydrochloric acid, orthophosphoric acid, carboxylic acids such as tartaric acid, citric acid, lactic acid, and sulphonic acids.

The dyeing composition in accordance with the invention may be provided in various forms, such as in the form of liquids, powders, creams, gels, optionally pressurized, or in any other form appropriate for dyeing keratinous fibers, and in particular human hair.

Concrete examples illustrating the invention are indicated below without however exhibiting a limiting character.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

### EXAMPLES

The following dyeing compositions were prepared:

(expressed in grams)

| | EXAMPLE | EXAMPLE |
|---|---|---|
| | 1 | 2 |
| | invention | prior art |
| 1-(4-Aminophenyl)pyrrolidine dihydrochloride | 0.47 | |
| (oxidation base according to the invention) | | |
| 2,4-Diamino-1-(β-hydroxyethyloxy)-benzene | 0.482 | 0.482 |
| dihydrochioride (coupler) | | |
| N, N-bis(β-Hydroxyethyl)-para- | | 0.392 |
| phenylendediamine sulphate | | |
| (oxidation base according to the prior | | |
| art) | | |
| Cationic polymer comprising repeating units (W) | 1 g AS* | 1 g AS* |
| Dye carrier (*) | qs | qs |
| Demineralized water qs | 100 | 100 |

AS* denotes active substance

(*) Dye Carrier Composition

| | | |
|---|---|---|
| −C₈−C₁₀ alkyl polyglucoside in aqueous solution at 60%, | 3.24 | g AS* |
| sold under the name ORAMIX CG 110 ® | | |
| by the company SEPPIC | | |
| Ethanol | 18.0 | g |
| Benzyl alcohol | 1.8 | g |
| Polyethylene glycol 400 | 2.7 | g |
| Pentasodium salt of diethylenetriaminepentaacetic acid | 0.43 | g AS* |
| in aqueous solution at 40%, sold under the | | |
| name DISSOLUINE D-40 ® by the company | | |
| AKZO | | |
| Sodium metabisulphite | 0.205 | g |
| Aqueous ammonia containing 20.5% of NH₃ | 10.0 | g |

AS* denotes active substance

At the time of use, each of the dyeing compositions described above were combined, weight for weight, with a solution of hydrogen peroxide at 20 volumes (6% by weight).

The combinations thus prepared were applied for 30 minutes to locks of natural grey hair which is 90% white. The locks were then rinsed, washed with a standard shampoo, rinsed again and then dried.

The color was then measured with a MINOLTA CM2002 colorimeter in the L*a*b* system.

In the L*a*b* system, the 3 parameters denote respectively the intensity (L*), the shade (a*) and the saturation (b*).

According to this system, the higher the value of L, the lighter or less intense the color. Conversely, the lower the value of L, the deeper or more intense the color.

The results have been grouped together in Table (I) below.

TABLE I

| EXAMPLE | L* |
|---|---|
| 2 | 25.91 |
| 1 | 22.27 |

Conclusion:

The dyeing with the combination according to the invention (1) is more intense than that of the prior art (2) [lower L value].

What is claimed is:

1. A composition for oxidation dyeing keratinous fibers comprising, in a medium suitable for dyeing:
(iii) at least one oxidation dye precursor chosen from 1-(4-aminophenyl)-pyrrolidines of formula (I) and acid addition salts thereof: 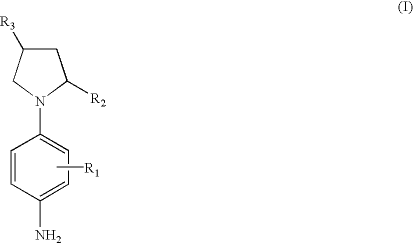
wherein:
R₁ is chosen from a hydrogen atom, C₁-C₆ alkyl groups, C₁-C₅ monohydroxyalkyl groups, and C₂-C₅ polyhydroxyalkyl groups,
R₂ is chosen from a hydrogen atom, a —CONH₂ group, C₁-C₅ monohydroxyalkyl groups, and C₂-C₅ polyhydroxyalkyl groups, and
R₃ is chosen from a hydrogen atom, and a hydroxyl group, and
(ii) at least one cationic polymer chosen from:
(1) homopolymers and copolymers comprising, as a constituent of the chain, at least one unit chosen from units formula (II): 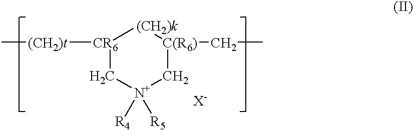
wherein:
k and t, which are identical or different, are each chosen from 0 and 1, provided that the sum of k+t is equal to 1,
R₄ and R₅, which are identical or different, are each chosen from (C₁-C₂₂) alkyl groups, (C₁-C₅)-hydroxyalkyl groups, and (C₁-C₄)amidoalkyl groups,
R₄ and R₅, together with the nitrogen cation to which they are commonly bonded, optionally form a cationic heterocyclic group chosen from a piperidinyl group and a morpholinyl group,
R₆, which are identical or different, are each chosen from a hydrogen atom and a methyl group, and
X⁻ is an anion;
(2) quaternary diammonium polymers comprising repeating units of formula (III): 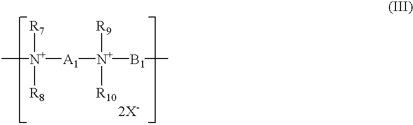
wherein:
R₇, R₈, R₉, and R₁₀, which are identical or different, are each chosen from C₁-C₂₀ aliphatic groups, C₃-C₂₀ alicyclic groups, C₇-C₂₀ arylaliphatic groups, and lower hydroxyalkyl groups,
at least two of said R₇, said R₈, said R₉, and said R₁₀, together with the nitrogen cations to which they are attached, optionally form at least one cationic heterocyclic ring optionally comprising an additional heteroatom other than nitrogen,
R₇, R₈, R₉, and R₁₀, which are identical or different, are each optionally chosen from linear and branched C₁-C₆ alkyl groups substituted with at least one group chosen from nitrile groups, ester groups, acyl groups, amide groups, —CO—O—R₁₁—D groups, and —CO—NH—R₁₁—D groups, wherein R₁₁ is chosen from alkylene groups and D is chosen from quaternary ammonium groups,
A₁ and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,
X⁻ is an anion,
said A₁, said R₇, and said R₉ optionally form a piperazine ring, together with the two nitrogen cations to which they are attached, and
provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is chosen from
—(CH₂)ₙ—CO—D—OC—(CH₂)ₙ— groups, wherein:
n is an integer ranging from 1 to 100,
D is chosen from:
a) glycol residues of formula: —O—Z—O—, wherein Z is chosen from linear and branched hydrocarbon groups and groups chosen from groups of formulae:
—(CH₂—CH₂—O)ₓ—CH₂—CH₂— and
—[CH₂—CH(CH₃)—O]_{y}—CH₂—CH(CH₃)—
wherein x and y, which are identical or different, are each chosen from integers ranging from 1 to 4 (in which case x and y represent a defined and unique degree of polymerization) and any number ranging from 1 to 4 (in which case x and y represent an average degree of polymerization),
b) bis-secondary diamine residues,
c) bis-primary diamine residues chosen from residues of formula: —NH—Y—NH—, wherein Y is chosen from linear and branched hydrocarbon groups and residues of formula
—CH₂—CH₂—S—S—CH₂—CH₂—, and
d) a ureylene group of formula: —NH—CO—NH—;
(3) quaternary diammonium polymers comprising repeating units of formula (IV): 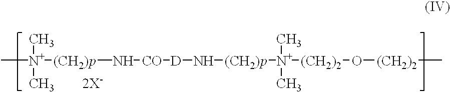
wherein:
p is an integer ranging from 1 to 6,
D is chosen from a direct bond and —(CH₂)ᵣ—CO— groups, wherein r is a number equal to 4 or 7, and
X⁻ is an anion;
(4) amine-containing silicones.

2. A composition according to claim 1, wherein said keratinous fibers are human keratinous fibers.

3. A composition according to claim 2, wherein said human keratinous fibers are human hair.

4. A composition according to claim 1, wherein said n of said —(CH₂)ₙ—CO—D—OC—(CH₂)ₙ— groups is an integer ranging from 1 to 50.

5. A composition according to claim 1, wherein said R₁, said R₂, and said R₃ are each a hydrogen atom.

6. A composition according to claim 1, wherein said R₁ and said R₃ are each a hydrogen atom and said R₂ is a —CH₂OH group.

7. A composition according to claim 1, wherein said R₁ is a hydrogen atom, said R₂ is a —CH₂OH group, and said R₃ is a hydroxyl group.

8. A composition according to claim 1, wherein said R₁ and said R₃ are each a hydrogen atom and said R₂ is a —CONH₂ group.

9. A composition according to claim 1, wherein said acid addition salts of said 1-(4-aminophenyl)pyrrolidines of formula (I) are chosen from hydrochlorides, hydrobromides, sulphates, tartrates, lactates, and acetates.

10. A composition according to claim 1, wherein said at least one oxidation dye precursor is present in said composition in an amount ranging from 0.001% to 10% by weight relative to the total weight of the composition.

11. A composition according to claim 10, wherein said at least one oxidation dye precursor is present in said composition in an amount ranging from 0.01% to 8% by weight relative to the total weight of the composition.

12. A composition according to claim 1, wherein said quaternary diammonium polymers comprising repeating units of formula (III) are chosen from cationic polymers comprising repeating units of formula (V): 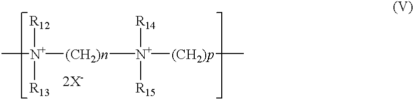
wherein
R₁₂, R₁₃, R₁₄, and R₁₅, which are identical or different, are each chosen from C₁-C₄ alkyl groups and C₁-C₄ hydroxyalkyl groups, and
n and p are each chosen from integers ranging from 2 to 20, and
X⁻ is an anion.

13. A composition according to claim 12, wherein said cationic polymers comprising repeating units of formula (V) are chosen from cationic polymers comprising repeating units of formula (W):

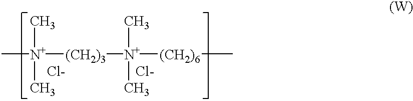

14. A composition according to claim 12, wherein said cationic polymers comprising repeating units of formula (V) are chosen from cationic polymers comprising repeating units of formula (U):

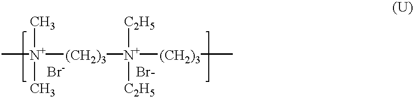

15. A composition according to claim 1, wherein said at least one cationic polymer is chosen from quaternary diammonium polymers comprising repeating units of formula (IV), wherein:
p is chosen from integers ranging from 1 to 6,
D is chosen from a direct bond and —(CH₂)ᵣ—CO— groups, wherein r is a number equal to 4 or 7, and
X⁻ is an anion chosen from anions derived from inorganic acids and anions derived from organic acids.

16. A composition according to claim 15, wherein said p is equal to 3, said D is a direct bond, and said X⁻ is a chlorine anion.

17. A composition according to claim 1, wherein said at least one amine-containing silicone is chosen from:
(i) polysiloxanes of formula (VI): 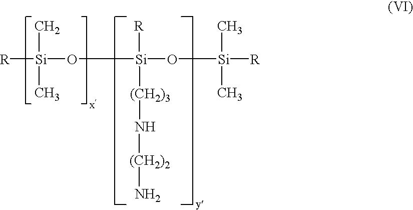
wherein:
R is a group chosen from a methyl group and a hydroxyl group, and
x′ and y′ are integers chosen such that the weight-average molecular weight of said polysiloxane ranges from 5,000 to 500,000;
(ii) aminosilicones of formula (VII):
(R¹)ₐ(T)₃₋ₐ-Si[OSi(T)₂]ₙ-[OSi(T)_{b}(R¹)_{2-b})]ₘ-O—Si(T)₃₋ₐ—(R¹)ₐ (VII)
wherein:
T is chosen from a hydrogen atom, a phenyl group, a hydroxyl group, and (C₁-C₈) alkyl groups,
a is an integer ranging from 0 to 3,
b is chosen from 0 and 1,
m and n are numbers such that the sum (n+m) ranges from 1 to 2,000,
n is chosen from a number ranging from 0 to 1,999,
m is chosen from a number ranging from 1 to 2,000,
R¹ is a monovalent group of formula —C_{q}H_{2q}L, wherein q is chosen from a number ranging from 2 to 8, and wherein L is an optionally quaternized amine group chosen from:
—N(R²)—CH₂—CH₂—N(R²)₂,
—N(R²)₂,
—N⁺(R²)₃Q⁻,
—N⁺(R²)(H)₂Q⁻,
—N⁺(R²)₂HQ⁻, and
—N(R²)—CH₂—CH₂—N⁺(R²)H₂Q⁻,
wherein
R², which are identical or different, are each chosen from a hydrogen atom, a phenyl group, a benzyl group, and (C₁-C₂₀) alkyl groups, and
Q⁻ is chosen from halide anions;
(iii) aminosilicones of formula (IX): 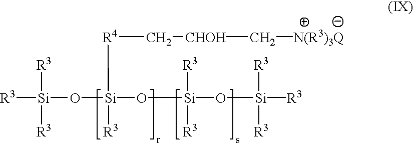
wherein:
R³, which are identical or different, are each chosen from (C₁-C₁₈) alkyl groups and (C₂-C₁₈) alkenyl groups,
R₄ is chosen from divalent (C₁-C₁₈) alkylene groups, and divalent (C₁-C₁₈) alkyleneoxy groups,
Q⁻ is chosen from halide anions,
r is a mean statistical value ranging from 2 to 20, and
s is a mean statistical value ranging from 20 to 200;
(iv) silicones of formula (X): 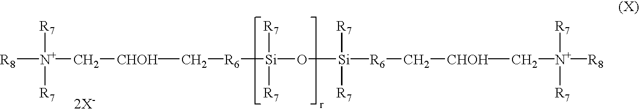
wherein:
R₆ is chosen from divalent (C₁-C₁₈) alkylene groups, and divalent (C₁-C₁₈) alkyleneoxy groups, wherein said R₆ is bonded to the Si by way of an SiC bond,
R₇, which are identical or different, are each chosen from (C₁-C₁₈) monovalent hydrocarbon-based groups, (C₂-C₁₈) alkenyl groups, and (C₅-C₆) rings,
R₈, which are identical or different, are each chosen from a hydrogen atom, (C₁-C₁₈) monovalent hydrocarbon-based groups, (C₂-C₁₈) alkenyl groups, and —R₆—NHCOR₇ groups, wherein said R₆ and said R₇ are defined above,
r is a mean statistical value ranging from 2 to 200, and
X⁻ is an anion.

18. A composition according to claim 1, wherein said at least one cationic polymer is chosen from trimethylsilylamodimethicones of formula (VIII): 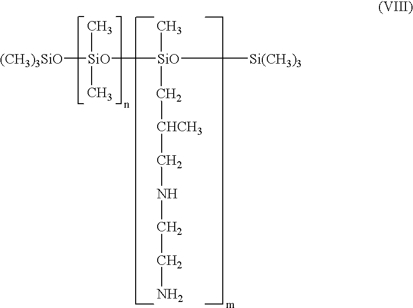
wherein:
m and n are numbers such that the sum (n+m) ranges from 1 to 2,000,
n is chosen from a number ranging from 0 to 1,999, and
m is chosen from a number ranging from 1 to 2,000.

19. A composition according to claim 1, wherein said at least one cationic polymer is chosen from polysiloxanes of formula (VI): 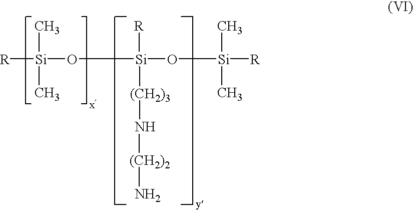
wherein:
R is a group chosen from a methyl group and a hydroxyl group, and
x′ and y′ are integers chosen such that the weight-average molecular weight of said polysiloxane ranges from 5,000 to 500,000;
and wherein said composition further comprises:
(i) trimethylcetylammonium chloride, and
(ii) at least one compound of formula: C₁₃H₂₇—(OC₂H₄)₁₂—OH.

20. A composition according to claim 18, wherein said composition further comprises:
(i) at least one nonionic surfactant of formula: C₈H₁₇—C₆H₄—(OCH₂CH₂)₄₀—OH,
(ii) at least one nonionic surfactant of formula: C₁₂H₂₅—(OCH₂—CH₂)₆—OH, and
(iii) propylene glycol.

21. A composition according to claim 1, wherein said at least one cationic polymer is present in said composition in an amount ranging from 0.01% to 10% by weight relative to the total weight of the composition.

22. A composition according to claim 21, wherein said at least one cationic polymer is present in said composition in an amount ranging from 0.05% to 5% by weight relative to the total weight of the composition.

23. A composition according to claim 22, wherein said at least one cationic polymer is present in said composition in an amount ranging from 0.1% to 3% by weight relative to the total weight of the composition.

24. A composition according to claim 1 further comprising at least one coupler.

25. A composition according to claim 24, wherein said at least one coupler is chosen from meta-phenylenediamines, meta-aminophenols, meta-diphenols, naphthols, heterocyclic couplers, and acid addition salts of any of the foregoing compounds.

26. A composition according to claim 24, wherein said at least one coupler is chosen from 2,4-diamino-1-(β-hydroxyethyloxy)benzene, 2-methyl-5-aminophenol, 5-N-(β-hydroxyethyl) amino-2-methylphenol, 3-aminophenol, 1,3-dihydroxybenzene, 1,3-dihydroxy-2-methylbenzene, 4-chloro-1,3-dihydroxy-benzene, 2-amino-4-(β-hydroxyethylamino)-1-methoxy-benzene, 1,3-diaminobenzene, 1,3-bis(2,4-diamino-phenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxy-indoline, 2,6-dihydroxy-4-methylpyridine, 1-H-3-methyl-pyrazol-5-one, 1-phenyl-3-methylpyrazol-5-one, 2-amino-3-hydroxypyridine, 3,6-dimethylpyrazolo[3,2-c]-1,2,4-triazole, 2,6-dimethylpyrazolo[1,5-b]-1,2,4-triazole and acid addition salts of any of the foregoing compounds.

27. A composition according to claim 24, wherein said at least one coupler is present in said composition in an amount ranging from 0.0001% to 15% by weight relative to the total weight of the composition.

28. A composition according to claim 1 further comprising at least one oxidation base, other than said at least one oxidation dye precursor, in an amount ranging from 0.0001% to 15% by weight relative to the total weight of said composition.

29. A composition according to claim 1 further comprising at least one direct dye in an amount ranging from 0.001% to 20% by weight relative to the total weight of said composition.

30. A composition according to claim 1 further comprising at least one agent chosen from reducing agents and antioxidants, wherein said at least one agent is present in said composition in an amount ranging from 0.05% to 1.5% by weight relative to the total weight of said composition.

31. A composition for oxidation dyeing keratinous fibers comprising, in a medium suitable for dyeing:
(i) at least one oxidation dye precursor chosen from 1-(4-aminophenyl)-pyrrolidines of formula (I) and acid addition salts thereof: 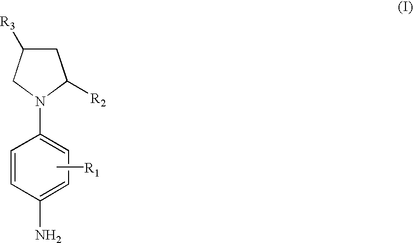
wherein:
R₁ is chosen from a hydrogen atom, C₁-C₆ alkyl groups, C₁-C₅ monohydroxyalkyl groups, and C₂-C₅ polyhydroxyalkyl groups,
R₂ is chosen from a hydrogen atom, a —CONH₂ group, C₁-C₅ monohydroxyalkyl groups, and C₂-C₅ polyhydroxyalkyl groups, and
R₃ is chosen from a hydrogen atom, and a hydroxyl group, and
(ii) at least one cationic polymer chosen from:
(1) homopolymers and copolymers comprising, as a constituent of the chain, at least one unit chosen from units formula (II): 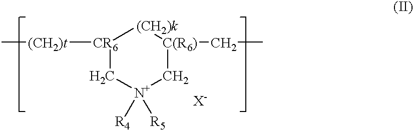
wherein:
k and t, which are identical or different, are each chosen from 0 and 1, provided that the sum of k+t is equal to 1,
R₄ and R₅, which are identical or different, are each chosen from (C₁-C₂₂) alkyl groups, (C₁-C₅)-hydroxyalkyl groups, and (C₁-C₄)amidoalkyl groups,
R₄ and R₅, together with the nitrogen cation to which they are commonly bonded, optionally form a cationic heterocyclic group chosen from a piperidinyl group and a morpholinyl group,
R₆, which are identical or different, are each chosen from a hydrogen atom and a methyl group, and
X⁻ is an anion;
(2) quaternary diammonium polymers comprising repeating units of formula (III): 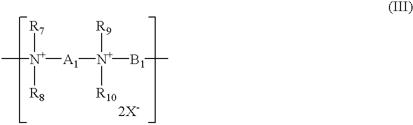
wherein:
R₇, R₈, R₉, and R₁₀, which are identical or different, are each chosen from C₁-C₂₀ aliphatic groups, C₃-C₂₀ alicyclic groups, C₇-C₂₀ arylaliphatic groups, and lower hydroxyalkyl groups,
at least two of said R₇, said R₈, said R₉, and said R₁₀, together with the nitrogen cations to which they are attached, optionally form at least one cationic heterocyclic ring optionally comprising an additional heteroatom other than nitrogen,
R₇, R₈, R₉, and R₁₀, which are identical or different, optionally are each chosen from linear and branched C₁-C₆ alkyl groups substituted with at least one group chosen from nitrile groups, ester groups, acyl groups, amide groups, —CO—O—R₁₁—D groups, and —CO—NH—R₁₁—D groups, wherein R₁₁ is chosen from alkylene groups and D is chosen from quaternary ammonium groups,
A₁ and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,
X⁻ is an anion,
said A₁, said R₇, and said R₉ optionally form a piperazine ring, together with the two nitrogen cations to which they are attached, and
provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is additionally chosen from —(CH₂)ₙ—CO—D—OC—(CH₂)ₙ— groups, wherein:
n is an integer ranging from 1 to 100, and
D is chosen from:
a) glycol residues of formula: —O—Z—O—, wherein Z is chosen from linear and branched hydrocarbon groups and groups chosen from groups of formulae:
—(CH₂—CH₂—O)ₓ—CH₂—CH₂— and
—[CH₂—CH(CH₃)—O]_{y}—CH₂—CH(CH₃)—
wherein x and y, which are identical or different, are each chosen from integers ranging from 1 to 4 (in which case x and y represent a defined and unique degree of polymerization) and any number ranging from 1 to 4 (in which case x and y represent an average degree of polymerization),
b) bis-secondary diamine residues,
c) bis-primary diamine residues chosen from residues of formula: —NH—Y—NH—, wherein Y is chosen from linear and branched hydrocarbon groups and residues of formula
—CH₂—CH₂—S—S—CH₂—CH₂—, and
d) a ureylene group of formula: —NH—CO—NH—;
(3) quaternary diammonium polymers comprising repeating units of formula (IV): 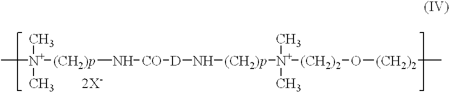
wherein:
p is an integer ranging from 1 to 6,
D is chosen from a direct bond and —(CH₂)ᵣ—CO— groups, wherein r is a number equal to 4 or 7, and
X⁻ is an anion;
(4) amine-containing silicones, and
(iii) at least one oxidizing agent.

32. A composition according to claim 31, wherein said at least one oxidizing agent is chosen from hydrogen peroxide, urea peroxide, alkali metal bromates, alkali metal ferricyanides, persalts, and oxidation-reduction enzymes

33. A composition according to claim 32, wherein said at least one oxidizing agent is chosen from laccases, peroxidases and 2-electron oxidoreductases, where appropriate in the presence of their respective donor or cofactor.

34. A composition according to claim 32, wherein said at least one oxidizing agent is hydrogen peroxide.

35. A composition according to claim 32, wherein said at least one oxidizing agent comprises a solution of hydrogen peroxide with a titre ranging from 1 to 40 in volume.

36. A composition according to claim 1, wherein said composition for oxidation dyeing keratinous fibers has a pH ranging from 3 to 12.

37. A composition according to claim 31, wherein said composition for oxidation dyeing keratinous fibers has a pH ranging from 3 to 12.

38. A composition according to claim 31 further comprising at least one surfactant chosen from anionic surfactants, cationic surfactants, nonionic surfactants, and amphoteric surfactants.

39. A composition according to claim 38, wherein said at least one surfactant is present in said composition in an amount ranging from 0.1% to 20% by weight relative to the total weight of said composition.

40. A method for oxidation dyeing keratinous fibers comprising:
(A) applying to said fibers at least one dyeing composition comprising, in a medium suitable for dyeing:
(i) at least one oxidation dye precursor chosen from 1-(4-aminophenyl)-pyrrolidines of formula (I) and acid addition salts thereof: 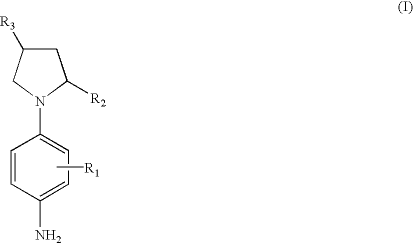
wherein:
R₁ is chosen from a hydrogen atom, C₁-C₆ alkyl groups, C₁-C₅ monohydroxyalkyl groups, and C₂-C₅ polyhydroxyalkyl groups,
R₂ is chosen from a hydrogen atom, a —CONH₂ group, C₁-C₅ monohydroxyalkyl groups, and C₂-C₅ polyhydroxyalkyl groups, and
R₃ is chosen from a hydrogen atom, and a hydroxyl group, and optionally comprising:
(ii) at least one cationic polymer chosen from:
(1) homopolymers and copolymers comprising, as a constituent of the chain, at least one unit chosen from units formula (II): 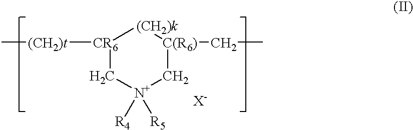
wherein:
k and t, which are identical or different, are each chosen from 0 and 1, provided that the sum of k+t is equal to 1,
R₄ and R₅, which are identical or different, are each chosen from (C₁-C₂₂) alkyl groups, (C₁-C₅)-hydroxyalkyl groups, and (C₁-C₄)amidoalkyl groups,
R₄ and R₅, together with the nitrogen cation to which they are commonly bonded, may optionally form a cationic heterocyclic group chosen from a piperidinyl group and a morpholinyl group,
R₆, which are identical or different, are each chosen from a hydrogen atom and a methyl group, and
X⁻ is an anion;
(2) quaternary diammonium polymers comprising repeating units of formula (III): 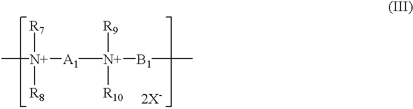
wherein:
R₇, R₈, R₉, and R₁₀, which are identical or different, are each chosen from C₁-C₂₀ aliphatic groups, C₃-C₂₀ alicyclic groups, C₇-C₂₀ arylaliphatic groups, and lower hydroxyalkyl groups,
at least two of said R₇, said R₈, said R₉, and said R₁₀, together with the nitrogen cations to which they are attached, optionally form at least one cationic heterocyclic ring optionally comprising an additional heteroatom other than nitrogen,
R₇, R₈, R₉, and R₁₀, which are identical or different, optionally are each chosen from linear and branched C₁-C₆ alkyl groups substituted with at least one group chosen from nitrile groups, ester groups, acyl groups, amide groups, —CO—O—R₁₁—D groups, and —CO—NH—R₁₁—D groups, wherein R₁₁ is chosen from alkylene groups and D is chosen from quaternary ammonium groups,
A₁ and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,
X⁻ is an anion,
said A₁, said R₇, and said R₉ optionally form a piperazine ring, together with the two nitrogen cations to which they are attached, and
provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is additionally chosen from
—(CH₂)ₙ—CO—D—OC—(CH₂)ₙ— groups, wherein:
n is an integer ranging from 1 to 100, and
D is chosen from:
a) glycol residues of formula: —O—Z—O—, wherein Z is chosen from linear and branched hydrocarbon groups and groups chosen from groups of formulae:
—(CH₂—CH₂—O)ₓ—CH₂—CH₂— and
—[CH₂—CH(CH₃)—O]_{y}—CH₂—CH(CH₃)—
wherein x and y, which are identical or different, are each chosen from integers ranging from 1 to 4 (in which case x and y represent a defined and unique degree of polymerization) and any number ranging from 1 to 4 (in which case x and y represent an average degree of polymerization),
b) bis-secondary diamine residues,
c) bis-primary diamine residues chosen from residues of formula: —NH—Y—NH—, wherein Y is chosen from linear and branched hydrocarbon groups and residues of formula
—CH₂—CH₂—S—S—CH₂—CH₂—, and
d) a ureylene group of formula: —NH—CO—NH—;
(3) quaternary diammonium polymers comprising repeating units of formula (IV): 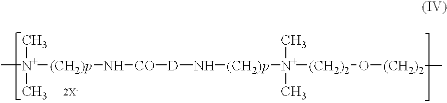
wherein:
p is an integer ranging from 1 to 6,
D is chosen from a direct bond and —(CH₂)ᵣ—CO— groups, wherein r is a number equal to 4 or 7, and
X⁻ is an anion;
(4) amine-containing silicones, and
(B) developing a color by applying to said fibers at least one oxidizing composition comprising:
at least one oxidizing agent and
optionally comprising said at least one cationic polymer as defined above,
wherein said at least one oxidizing composition is
applied to said fibers after combining, at the time of use, said at least one oxidizing composition with said at least one dyeing composition, or
applied to said fibers either simultaneously with or immediately after said at least one dyeing composition, without intermediate rinsing, and
(C) provided that said at least one cationic polymer is present in at least one of said at least one dyeing composition or said at least one oxidizing composition.

41. A method according to claim 40, wherein said keratinous fibers are human keratinous fibers.

42. A method according to claim 41, wherein said human keratinous fibers are human hair.

43. A method for oxidation dyeing keratinous fibers comprising:
(A) preparing at least one dyeing composition comprising, in a medium suitable for dyeing:
(i) at least one oxidation dye precursor chosen from 1-(4-aminophenyl)-pyrrolidines of formula (I) and acid addition salts thereof: 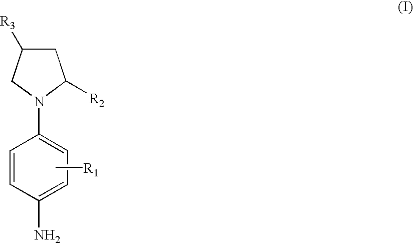
wherein:
R₁ is chosen from a hydrogen atom, C₁-C₆ alkyl groups, C₁-C₅ monohydroxyalkyl groups, and C₂-C₅ polyhydroxyalkyl groups,
R₂ is chosen from a hydrogen atom, a —CONH₂ group, C₁-C₅ monohydroxyalkyl groups, and C₂-C₅ polyhydroxyalkyl groups, and
R₃ is chosen from a hydrogen atom, and a hydroxyl group,
(ii) at least one cationic polymer chosen from:
(1) homopolymers and copolymers comprising, as a constituent of the chain, at least one unit chosen from units formula (II): 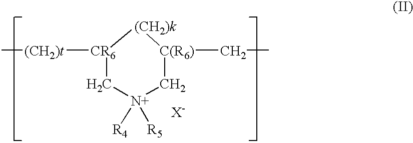
wherein:
k and t, which are identical or different, are each chosen from 0 and 1, provided that the sum of k+t is equal to 1,
R₄ and R₅, which are identical or different, are each chosen from (C₁-C₂₂) alkyl groups, (C₁-C₅)-hydroxyalkyl groups, and (C₁-C₄)amidoalkyl groups,
R₄ and R₅, together with the nitrogen cation to which they are commonly bonded, may optionally form a cationic heterocyclic group chosen from a piperidinyl group and a morpholinyl group,
R₆, which are identical or different, are each chosen from a hydrogen atom and a methyl group, and
X⁻ is an anion;
(2) quaternary diammonium polymers comprising repeating units of formula (III): 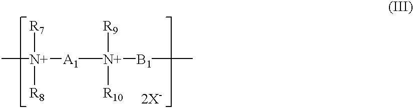
wherein:
R₇, R₈, R₉, and R₁₀, which are identical or different, are each chosen from C₁-C₂₀ aliphatic groups, C₃-C₂₀ alicyclic groups, C₇-C₂₀ arylaliphatic groups, and lower hydroxyalkyl groups,
at least two of said R₇, said R₈, said R₉, and said R₁₀, together with the nitrogen cations to which they are attached, optionally form at least one cationic heterocyclic ring optionally comprising an additional heteroatom other than nitrogen,
R₇, R₈, R₉, and R₁₀, which are identical or different, optionally are each chosen from linear and branched C₁-C₆ alkyl groups substituted with at least one group chosen from nitrile groups, ester groups, acyl groups, amide groups, —CO—O—R₁₁—D groups, and —CO—NH—R₁₁—D groups, wherein R₁₁ is chosen from alkylene groups and D is chosen from quaternary ammonium groups,
A₁ and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,
X⁻ is an anion,
said A₁, said R₇, and said R₉ optionally form a piperazine ring, together with the two nitrogen cations to which they are attached, and
provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is additionally chosen from
—(CH₂)ₙ—CO—D—OC—(CH₂)ₙ— groups, wherein:
n is an integer ranging from 1 to 100, and
D is chosen from:
a) glycol residues of formula: —O—Z—O—, wherein Z is chosen from linear and branched hydrocarbon groups and groups chosen from groups of formulae:
—(CH₂—CH₂—O)ₓ—CH₂—CH₂— and
—[CH₂—CH(CH₃)—O]_{y}—CH₂—CH(CH₃)—
wherein x and y, which are identical or different, are each chosen from integers ranging from 1 to 4 (in which case x and y represent a defined and unique degree of polymerization) and any number ranging from 1 to 4 (in which case x and y represent an average degree of polymerization),
b) bis-secondary diamine residues,
c) bis-primary diamine residues chosen from residues of formula: —NH—Y—NH—, wherein Y is chosen from linear and branched hydrocarbon groups and residues of formula
—CH₂—CH₂—S—S—CH₂—CH₂—, and
d) a ureylene group of formula: —NH—CO—NH—;
(3) quaternary diammonium polymers comprising repeating units of formula (IV): 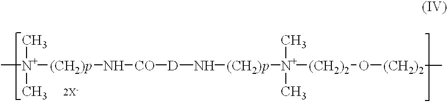
wherein:
p is an integer ranging from 1 to 6,
D is chosen from a direct bond and —(CH₂)ᵣ—CO— groups, wherein r is a number equal to 4 or 7, and
X⁻ is an anion;
(4) amine-containing silicones, and
(iii) at least one oxidizing agent,
wherein said at least one dyeing composition is prepared, at the time of use, by combining (i), (ii), and (iii) above,
(B) developing a color by applying said at least one dyeing composition prepared in (A) above to said fibers,
(C) leaving said at least one dyeing composition prepared in (A) above on said fibers for a time ranging from 1 to 60 minutes,
(D) rinsing said fibers, optionally shampooing said fibers, and optionally further rinsing said fibers, and
(E) drying said fibers.

44. A method according to claim 43, wherein said keratinous fibers are human keratinous fibers.

45. A method according to claim 44, wherein said human keratinous fibers are human hair.

46. A method according to claim 43, wherein said leaving time is a time ranging from 10 to 45 minutes.

47. A method according to claim 45, wherein said human hair is chosen from wet human hair and dry human hair.

48. A kit for oxidation dyeing keratinous fibers comprising at least two compartments, wherein:
(A) a first compartment comprises at least one dyeing composition comprising, in a medium suitable for dyeing:
(i) at least one oxidation dye precursor chosen from 1-(4-aminophenyl)-pyrrolidines of formula (I) and acid addition salts thereof: 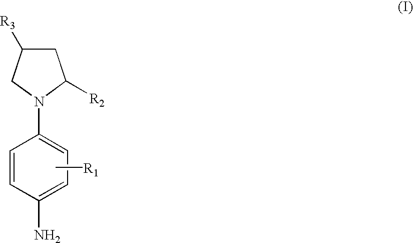
wherein:
R₁ is chosen from a hydrogen atom, C₁-C₆ alkyl groups, C₁-C₅ monohydroxyalkyl groups, and C₂-C₅ polyhydroxyalkyl groups,
R₂ is chosen from a hydrogen atom, a —CONH₂ group, C₁-C₅ monohydroxyalkyl groups, and C₂-C₅ polyhydroxyalkyl groups, and
R₃ is chosen from a hydrogen atom, and a hydroxyl group,
and optionally comprising:
(ii) at least one cationic polymer chosen from:
(1) homopolymers and copolymers comprising, as a constituent of the chain, at least one unit chosen from units formula (II): 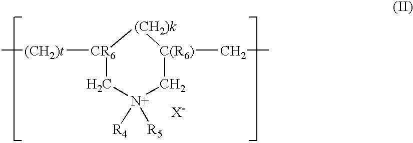
wherein:
k and t, which are identical or different, are each chosen from 0 and 1, provided that the sum of k+t is equal to 1,
R₄ and R₅, which are identical or different, are each chosen from (C₁-C₂₂) alkyl groups, (C₁-C₅)-hydroxyalkyl groups, and (C₁-C₄)amidoalkyl groups,
R₄ and R₅, together with the nitrogen cation to which they are commonly bonded, may optionally form a cationic heterocyclic group chosen from a piperidinyl group and a morpholinyl group,
R₆, which are identical or different, are each chosen from a hydrogen atom and a methyl group, and
X⁻ is an anion;
(2) quaternary diammonium polymers comprising repeating units of formula (III): 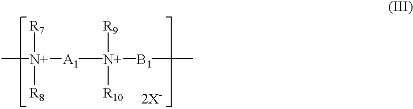
wherein:
R₇, R₈, R₉, and R₁₀, which are identical or different, are each chosen from C₁-C₂₀ aliphatic groups, C₃-C₂₀ alicyclic groups, C₇-C₂₀ arylaliphatic groups, and lower hydroxyalkyl groups,
at least two of said R₇, said R₈, said R₉, and said R₁₀, together with the nitrogen cations to which they are attached, optionally form at least one cationic heterocyclic ring optionally comprising an additional heteroatom other than nitrogen,
R₇, R₈, R₉, and R₁₀, which are identical or different, optionally are each chosen from linear and branched C₁-C₆ alkyl groups substituted with at least one group chosen from nitrile groups, ester groups, acyl groups, amide groups, —CO—O—R₁₁—D groups, and —CO—NH—R₁₁—D groups, wherein R₁₁ is chosen from alkylene groups and D is chosen from quaternary ammonium groups,
A₁ and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,
X⁻ is an anion,
said A₁, said R₇, and said R₉ optionally form a piperazine ring, together with the two nitrogen cations to which they are attached, and
provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is additionally chosen from
—(CH₂)ₙ—CO—D—OC—(CH₂)ₙ— groups, wherein:
n is an integer ranging from 1 to 100, and
D is chosen from:
a) glycol residues of formula: —O—Z—O—, wherein Z is chosen from linear and branched hydrocarbon groups and groups chosen from groups of formulae:
—(CH₂—CH₂—O)ₓ—CH₂—CH₂— and
—[CH₂—CH(CH₃)—O]_{y}—CH₂—CH(CH₃)—
wherein x and y, which are identical or different, are each chosen from integers ranging from 1 to 4 (in which case x and y represent a defined and unique degree of polymerization) and any number ranging from 1 to 4 (in which case x and y represent an average degree of polymerization),
b) bis-secondary diamine residues,
c) bis-primary diamine residues chosen from residues of formula: —NH—Y—NH—, wherein Y is chosen from linear and branched hydrocarbon groups and residues of formula
—CH₂—CH₂—S—S—CH₂—CH₂—, and
d) a ureylene group of formula: —NH—CO—NH—;
(3) quaternary diammonium polymers comprising repeating units of formula (IV): 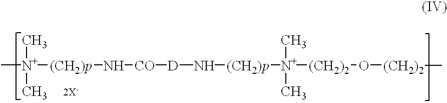
wherein:
p is an integer ranging from 1 to 6,
D is chosen from a direct bond and —(CH₂)ᵣ—CO— groups, wherein r is a number equal to 4 or 7, and
X⁻ is an anion; and
(4) amine-containing silicones, and
(B) a second compartment comprises at least one oxidizing agent and optionally comprises said at least one cationic polymer as defined above, and
(C) provided that said at least one cationic polymer is present in at least one of said first compartment or said second compartment.

49. A kit according to claim 48, wherein said keratinous fibers are human keratinous fibers.

50. A kit according to claim 49, wherein said human keratinous fibers are human hair.

51. A kit for oxidation dyeing keratinous fibers comprising at least three compartments, wherein:
(A) a first compartment comprises at least one dyeing composition comprising, in a medium suitable for dyeing, at least one oxidation dye precursor chosen from 1-(4-aminophenyl)-pyrrolidines of formula (I) and acid addition salts thereof: 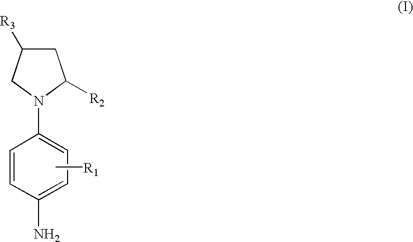
wherein:
R₁ is chosen from a hydrogen atom, C₁-C₆ alkyl groups, C₁-C₅ monohydroxyalkyl groups, and C₂-C₅ polyhydroxyalkyl groups,
R₂ is chosen from a hydrogen atom, a —CONH₂ group, C₁-C₅ monohydroxyalkyl groups, and C₂-C₅ polyhydroxyalkyl groups, and
R₃ is chosen from a hydrogen atom, and a hydroxyl group,
(B) a second compartment comprises at least one cationic polymer chosen from:
(1) homopolymers and copolymers comprising, as a constituent of the chain, at least one unit chosen from units formula (II): 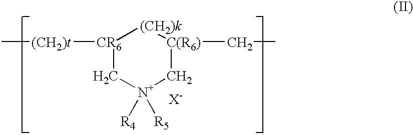
wherein:
k and t, which are identical or different, are each chosen from 0 and 1, with the proviso that the sum of k+t is equal to 1,
R₄ and R₅, which are identical or different, are each chosen from (C₁-C₂₂) alkyl groups, (C₁-C₅)-hydroxyalkyl groups, and (C₁-C₄)amidoalkyl groups,
R₄ and R₅, together with the nitrogen cation to which they are commonly bonded, may optionally form a cationic heterocyclic group chosen from a piperidinyl group and a morpholinyl group,
R₆, which are identical or different, are each chosen from a hydrogen atom and a methyl group, and
X⁻ is an anion;
(2) quaternary diammonium polymers comprising repeating units of formula (III): 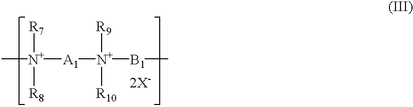
wherein:
R₇, R₈, R₉, and R₁₀, which are identical or different, are each chosen from C₁-C₂₀ aliphatic groups, C₃-C₂₀ alicyclic groups, C₇-C₂₀ arylaliphatic groups, and lower hydroxyalkyl groups,
at least two of said R₇, said R₈, said R₉, and said R₁₀, together with the nitrogen cations to which they are attached, optionally form at least one cationic heterocyclic ring optionally comprising an additional heteroatom other than nitrogen,
R₇, R₈, R₉, and R₁₀, which are identical or different, optionally are each chosen from linear and branched C₁-C₆ alkyl groups substituted with at least one group chosen from nitrile groups, ester groups, acyl groups, amide groups, —CO—O—R₁₁—D groups, and —CO—NH—R₁₁—D groups, wherein R₁₁ is chosen from alkylene groups and D is chosen from quaternary ammonium groups,
A₁ and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,
X⁻ is an anion,
said A₁, said R₇, and said R₉ optionally form a piperazine ring, together with the two nitrogen cations to which they are attached, and
provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is additionally chosen from
—(CH₂)ₙ—CO—D—OC—(CH₂)ₙ— groups, wherein:
n is an integer ranging from 1 to 100, and
D is chosen from:
a) glycol residues of formula: —O—Z—O—, wherein Z is chosen from linear and branched hydrocarbon groups and groups chosen from groups of formulae:
—(CH₂—CH₂—O)ₓ—CH₂—CH₂— and
—[CH₂—CH(CH₃)—O]_{y}—CH₂—CH(CH₃)—
wherein x and y, which are identical or different, are each chosen from integers ranging from 1 to 4 (in which case x and y represent a defined and unique degree of polymerization) and any number ranging from 1 to 4 (in which case x and y represent an average degree of polymerization),
b) bis-secondary diamine residues,
c) bis-primary diamine residues chosen from residues of formula: —NH—Y—NH—, wherein Y is chosen from linear and branched hydrocarbon groups and residues of formula —CH₂—CH₂—S—S—CH₂—CH₂—, and
d) a ureylene group of formula: —NH—CO—NH—;
(3) quaternary diammonium polymers comprising repeating units of formula (IV): 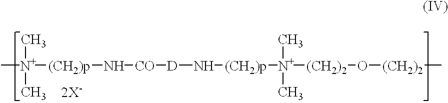
wherein:
p is an integer ranging from 1 to 6,
D is chosen from a direct bond and —(CH₂)ᵣ—CO— groups, wherein r is a number equal to 4 or 7, and
X⁻ is an anion; and
(4) amine-containing silicones, and
(C) a third compartment comprises at least one oxidizing agent.

52. A kit according to claim 51, wherein said keratinous fibers are human keratinous fibers.

53. A kit according to claim 52, wherein said human keratinous fibers are human hair.
